(19) United States
(12) Patent Application Publication
(10) Pub. No.: US 2022/0389006 A1
(43) Pub. Date: Dec. 8, 2022

(54) SALTS AND FORMS OF AN ESTROGEN RECEPTOR MODULATOR

(71) Applicant: Recurium IP Holdings, LLC, San Diego, CA (US)

(72) Inventors: Peter Qinhua Huang, San Diego, CA (US); Sayee Gajanan Hegde, San Diego, CA (US); Kevin Duane Bunker, Escondido, CA (US); John Knight, San Diego, CA (US); Deborah Helen Slee, Encinitas, CA (US)

(21) Appl. No.: 17/755,562

(22) PCT Filed: Nov. 2, 2020
(86) PCT No.: PCT/US2020/058526

§ 371 Date: May 2, 2022

Related U.S. Application Data
(60) Provisional application No. 62/930,153, filed on Nov. 4, 2019.

(51) Int. Cl. C07D 471/04

(52) U.S. Cl. CPC C07D 471/04; C07B 2200/13

(57) ABSTRACT

Salts of Compound A and forms described herein are estrogen receptor alpha modulators. Such salts and/or forms can be are useful for treating diseases or conditions that are estrogen receptor alpha dependent and/or estrogen receptor alpha mediated, including conditions characterized by excessive cellular proliferation, such as breast cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 62/930,153, filed Nov. 4, 2019.

### FIELD

The present application relates to compounds, salts and salt forms that are estrogen receptor alpha modulators and methods of using them to treat conditions characterized by excessive cellular proliferation, such as cancer.

### DESCRIPTION

Many cancer cells express estrogen receptors (ERs) and have growth characteristics that are modulated by estrogen. A number of breast cancer drug therapies have been developed that target ERs. In many cases the drugs are selective estrogen receptor modulators (SERMs) that have agonistic and/or antagonistic effects on ERs. For example, fulvestrant is a drug that is used for the treatment of metastatic breast cancer. It has antagonistic effects on ER-alpha and is considered a selective estrogen receptor alpha degrader (SERD). Fulvestrant has the following chemical structure:

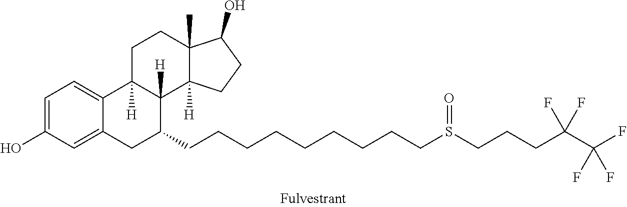

At this time the only SERD approved for the treatment of breast cancer in the United States is fulvestrant. However, the clinical efficacy of fulvestrant is limited and fulvestrant has to be dosed via intramuscular injection. A number of orally dosed SERDs are currently in clinical development (e.g., AZD9496, RAD1901, LSZ102, H3B-9545, GIT48, D-0502, SHR9549, lasofoxifene, ARV-378, GDC-9545, SAR439859 and AZD9833), but at this time no oral SERD has been approved for the treatment of breast cancer in the United States (see De Savi, C. et al. publication referenced above). Thus, there remains a long-felt need for well tolerated orally dosed SERDs or SERMs that are useful in the study and the treatment of proliferative disorders, such as breast cancer, that have growth characteristics that are modulated by estrogen.

### SUMMARY

Some embodiments disclosed herein relate to a pharmaceutically acceptable salt of Compound A, wherein the pharmaceutically acceptable salt is a hydrosulfate salt of Compound A. Other embodiments disclosed herein relate to a pharmaceutically acceptable salt of Compound A, wherein the pharmaceutically acceptable salt is a sulfate salt of Compound A. Still other embodiments disclosed herein related to one or more salt forms of Compound A. In some embodiments, a pharmaceutically acceptable salt of Compound A can be crystalline. In some embodiments, a crystalline pharmaceutically acceptable salt of Compound A can exist as a polymorph.

Still other embodiments disclosed herein relate to a pharmaceutical composition that can include an effective amount of one or more salts Compound A and/or one or more salt forms of Compound A, and a pharmaceutically acceptable carrier, diluent, excipient or combination thereof.

Yet still other embodiments disclosed herein relate to a method of treatment that can include identifying a subject that is in need of treatment for a disease or condition that is estrogen receptor alpha dependent and/or estrogen receptor alpha mediated; and administering to said subject an effective amount of one or more salts Compound A and/or one or more salt forms of Compound A, or a pharmaceutical composition that can include an effective amount of one or more salts Compound A and/or one or more salt forms of Compound A. In some embodiments, the disease or condition can be selected from a breast cancer and a gynecological cancer. In some embodiments, the disease or condition can be selected from breast cancer, endometrial cancer, ovarian cancer and cervical cancer.

Some embodiments disclosed herein relate to the use of one or more salts Compound A and/or one or more salt forms of Compound A, or a pharmaceutical composition that can include an effective amount of one or more salts Compound A and/or one or more salt forms of Compound A, for use in the treatment of a disease or condition that is estrogen receptor alpha dependent and/or estrogen receptor alpha mediated. Other embodiments disclosed herein relate to the use of one or more salts Compound A and/or one or more salt forms of Compound A, or a pharmaceutical composition that can include an effective amount of one or more salts Compound A and/or one or more salt forms of Compound A, in the preparation of a medicament for use in the treatment of a disease or condition that is estrogen receptor alpha dependent and/or estrogen receptor alpha mediated.

These and other embodiments are described in greater detail below.

### DRAWINGS

FIG. 1 provides a representative X-ray powder diffraction (XRPD) pattern of Form A.

FIG. 2 provides a representative DSC thermogram of Form A.

FIG. 3 provides a representative ¹H NMR spectrum of Form A, wherein the solvent is CD₃OD.

FIG. 4 provides a representative ¹H NMR spectrum of Form A, wherein the solvent is DMSO-d₆.

FIG. 5 provides a representative XRPD pattern of Form C.

FIG. 6A provides a representative DSC thermogram of Form C. FIG. 6B provides a second representative DSC thermogram along with a TGA thermogram of Form C.

FIG. 7 provides a representative ¹H NMR spectrum of Form C, wherein the solvent is DMSO-d₆.

FIG. 8 provides a representative XRPD pattern of Form D.

FIG. 9 provides a representative DSC thermogram of Form D.

FIG. 10 provides a representative XRPD pattern of Form E.

FIG. 11 provides a representative DSC thermogram of Form E.

FIG. 12 provides a representative XRPD pattern of Form D, Form E and free base Form I, wherein the XRPD pattern of free base Form I is used as a reference.

FIG. 13 provides a representative XRPD pattern of a first HCl salt of Compound A.

FIG. 14 provides a representative XRPD pattern of a second HCl salt of Compound A.

FIG. 15 provides a representative XRPD pattern of a first HCl salt of Compound A (HCl salt Form A), a second HCl salt of Compound A (HCl salt Form B) and free base Form I, wherein the XRPD pattern of free base Form I is used as a reference.

FIG. 16 provides a representative DSC thermogram along with a TGA thermogram of a first HCl salt of Compound A (HCl salt of Form A).

FIG. 17 provides a representative XRPD pattern of a citrate salt of Compound A and free base Form I, wherein the XRPD pattern of free base Form I is used as a reference.

FIG. 18 provides a representative DSC thermogram along with a TGA thermogram of a citrate salt of Compound A.

FIG. 19 provides a representative XRPD pattern of a first mesylate salt of Compound A.

FIG. 20 provides a representative XRPD pattern of a second mesylate salt of Compound A.

FIG. 21 provides a representative XRPD pattern of a first mesylate salt of Compound A (mesylate salt of Form A), a second mesylate salt of Compound A (mesylate salt of Form B) and free base Form I, wherein the XRPD pattern of free base Form I is used as a reference.

FIG. 22 provides a representative DSC thermogram along with a TGA thermogram of a first mesylate salt of Compound A (mesylate salt Form A).

FIG. 23 provides a representative DSC thermogram along with a TGA thermogram of a second mesylate salt of Compound A (mesylate salt Form B).

FIG. 24 provides a representative XRPD pattern of a besylate salt of Compound A and free base Form I, wherein the XRPD patter of free base Form I is used as a reference.

FIG. 25 provides a representative DSC thermogram along with a TGA thermogram of a besylate salt of Compound A.

FIG. 26 provides a representative XRPD pattern of a choline salt of Compound A.

FIG. 27 provides a representative XRPD pattern of a choline salt of Compound A and free base Form I, wherein the XRPD patter of free base Form I is used as a reference.

FIG. 28 provides a second representative DSC thermogram along with a TGA thermogram of a choline salt of Compound A.

FIG. 29 provides a representative DSC thermogram along with a TGA thermogram of free base of Form I.

FIG. 30 provides a representative XRPD pattern of free base Form I initially, after 1 day, after 3 days and after 7 days.

FIG. 31 provides a representative XRPD pattern of Form A (before heating, after heating to 100° C. and after heating to 150° C.).

FIG. 32A provides a representative DSC thermogram and a TGA thermogram of Form A after being heated to 100° C. FIG. 32B provides a representative DSC thermogram and a TGA thermogram of Form A after being heated to 150° C.

FIG. 33 provides a representative ¹H NMR spectra of Form A (before heating, after heating to 100° C. and after heating to 150° C.).

FIG. 34 provides a TGA thermogram for (1) Form A for a sample heated to 150° C., and (2) a sample initially dried under vacuum for 3 hours at 40° C.

FIG. 35A provides a representative XRPD pattern of Form A after one week (initial, 25° C./60% relative humidity and 50° C./75% relative humidity). FIG. 35B provides a representative XRPD pattern of Form C after one week (initial, 25° C./60% relative humidity and 50° C./75% relative humidity).

FIG. 36 provides a representative XRPD pattern of Form C prior to heating and after heating to 150° C., and a representative XRPD pattern of Form A.

FIG. 37 provides a representative XRPD pattern of a second HCl salt of Compound A (prior to sample preparation and after re-preparation of the sample).

FIG. 38 provides a representative XRPD of a first mesylate salt of Compound A and Form A both before and after grinding.

FIG. 39 provides a representative XRPD pattern of an oxalate salt of Compound A and free base Form I, wherein the XRPD patter of free base Form I is used as a reference.

FIG. 40 provides a representative XRPD pattern of each of Form A, Form B and free base Form I.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Unless otherwise specified, the term “crystalline” and related terms used herein, when used to describe a substance, component, product or form, mean that the substance, component, product or form is substantially crystalline, for example, as determined by X-ray diffraction. (see, e.g., Remington's Pharmaceutical Sciences, 20^{th} ed., Lippincott Williams & Wilkins, Philadelphia Pa., 173 (2000); The United States Pharmacopeia, 37^{th} ed., 503-509 (2014)).

As used herein, and unless otherwise specified, the terms “about” and “approximately,” when used in connection with doses, amounts or weight percents of ingredients of a composition or a dosage form, mean a dose, amount or weight percent that is recognized by one of ordinary skill in the art to provide a pharmacological effect equivalent to that obtained from the specified dose, amount, or weight percent. In some embodiments, the terms “about” and “approximately,” when used in this context, contemplate a dose, amount, or weight percent within 30%, within 20%, within 15%, within 10%, or within 5%, of the specified dose, amount or weight percent.

As used herein, and unless otherwise specified, the terms “about” and “approximately,” when used in connection with a numeric value or range of values which is provided to characterize a particular solid form, e.g., a specific temperature or temperature range (for example, that describes a melting, dehydration, desolvation or glass transition temperature); a mass change (for example, a mass change as a function of temperature or humidity); a solvent or water content (for example, mass or a percentage); or a peak position (for example, in analysis by, for example, IR or Raman spectroscopy or XRPD); indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies and dissolution studies. In some embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. In the context of molar ratios, “about” and “approximately” indicate that the numeric value or range of values may vary within 20%, 15%, 10%, 9%, 8%, 7%, 6%, 5%, 4%, 3%, 2%, 1.5%, 1%, 0.5%, or 0.25% of the recited value or range of values. It should be understood that the numerical values of the peaks of an X-ray powder diffraction pattern may vary from one machine to another, or from one sample to another, and so the values quoted are not to be construed as absolute, but with an allowable variability, such as ±0.2 degrees two theta (°20), or more. For example, in some embodiments, the value of an XRPD peak position may vary by up to ±0.2 degrees 2θ while still describing the particular XRPD peak.

As used herein, and unless otherwise specified, a solid form that is “substantially physically pure” is substantially free from other solid forms. In some embodiments, a crystal form that is substantially physically pure contains less than about 50%, 45%, 40%, 35%, 30%, 25%, 20%, 15%, 10%, 9%, 8%, 7%, 6%, 5%, 4%, 3%, 20%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In some embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains, In some embodiments, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% by weight of the other compound, solid form, or composition.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

As used herein, (E)-3-(4-((1R,3R)-2-(Bicyclo[1.1.1]pentan-1-yl)-3-methyl-2,3,4,9-tetrahydro-1H-pyrido[3,4-b]indol-1-yl)-3,5-difluorophenyl)acrylic acid is Compound A, which has the structure:

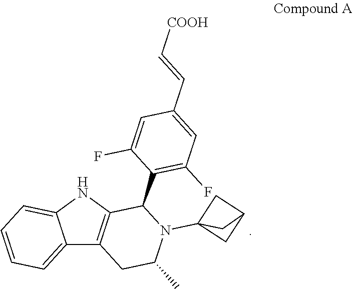 [SMILES: C[C@@H]1Cc2c([nH]c3ccccc23)[C@@H](c2c(F)cc(/C=C/C(=O)O)cc2F)N1C12CC(C1)C2]

Compound A is also referred to herein as the “free base of Compound A.” If there is an inconsistency between the name of Compound A and a structure of Compound A provided herein, then the structure of Compound A in this paragraph is what is meant for Compound A.

Some embodiments disclosed herein relate to a pharmaceutically acceptable salt of Compound A, wherein the pharmaceutically acceptable salt is a hydrosulfate salt of Compound A. Those skilled in the art understand that the hydrosulfate salt of Compound A has about a single molecule of Compound A for about a single molecule of hydrogen sulfate.

Other embodiments disclosed herein relate to a pharmaceutically acceptable salt of Compound A, wherein the pharmaceutically acceptable salt is a sulfate salt of Compound A. Those skilled in the art understand that the sulfate salt of Compound A has about two molecules of Compound A for about a single molecule of sulfate. Further, those skilled in the art understand that hydrogen sulfate and sulfate salts of Compound A are where one or more of the nitrogen atoms of Compound A can be protonated.

Still other embodiments disclosed herein relate to a pharmaceutically acceptable salt form of Compound A that can include the hydrosulfate (HSO₄⁻) salt of Compound A and the sulfate (SO₄²⁻) salt of Compound A.

Yet still other embodiments disclosed herein relate to a pharmaceutically acceptable salt form of Compound A that consists essentially of the hydrosulfate salt of Compound A and the sulfate salt of Compound A.

Various amounts of the hydrosulfate salt of Compound A and the sulfate salt of Compound A can be included in a pharmaceutically acceptable salt form described herein (for example, Form A and/or Form C). In some embodiments, the amount of the hydrosulfate salt of Compound A+the amount of the sulfate salt of Compound A can be ≥85% of the pharmaceutically acceptable salt form described herein (such as Form A and/or Form C). In other embodiments, the amount of the hydrosulfate salt of Compound A+the amount of the sulfate salt of Compound A can be ≥90% of the pharmaceutically acceptable salt form described herein (e.g., Form A and/or Form C). In still other embodiments, the amount of the hydrosulfate salt of Compound A+the amount of the sulfate salt of Compound A can be ≥95% of the pharmaceutically acceptable salt form described herein (for example, Form A and/or Form C). In yet still other embodiments, the amount of the hydrosulfate salt of Compound A+the amount of the sulfate salt of Compound A can be ≥98% of the pharmaceutically acceptable salt form described herein (such as Form A and/or Form C). In some embodiments, the amount of the hydrosulfate salt of Compound A+the amount of the sulfate salt of Compound A can be equal to 100% of the pharmaceutically acceptable salt form described herein (for example, Form A and/or Form C).

A variety of salt forms of Compound A can be obtained. In some embodiments, the salt form can be Form A. In other embodiments, the salt form can be Form C. The salt forms described herein can include the hydrosulfate salt of Compound A and/or the sulfate salt of Compound A. In some embodiments, a salt form described herein further can include the free base of Compound A.

In a salt form of Compound A, various amounts of the hydrogen sulfate salt of Compound A can be present. For example, the amount of hydrogen sulfate salt of Compound A that can be present in a salt form described herein (such as Form A and Form C) can be in the range of about 90% to 100%. In some embodiments, the amount of hydrogen sulfate salt of Compound A that can be present in a salt form described herein can be in the range of about 95% to about 100%. In other embodiments, the amount of hydrogen sulfate salt of Compound A that can be present in a salt form described herein can be in the range of about 98% to about 100%. In still other embodiments, the amount of hydrogen sulfate salt of Compound A that can be present in a salt form described herein can be in the range of about 95% to about 98%. When less than 100% of a salt form described herein is the hydrogen sulfate salt of Compound A, one or more of the components selected from the following can be present in the salt form (such as Form A and Form C): (1) the sulfate salt of Compound A, (2) the free base of Compound A, (3) a compound that is the result of the degradation of the hydrogen sulfate salt of Compound A, the degradation of the sulfate salt of Compound A and/or the degradation of the free base of Compound A, and (4) an impurity from the synthesis of the hydrogen sulfate salt of Compound A and/or the synthesis of the free base of Compound A.

In some embodiments, the amount of hydrogen sulfate salt of Compound A that can be present in Form A can be in the range of about 90% to about 98%. In other embodiments, the amount of hydrogen sulfate salt of Compound A that can be present in Form A can be in the range of about 95% to about 100%. In still other embodiments, the amount of hydrogen sulfate salt of Compound A that can be present in Form A can be in the range of about 98% to about 100%. In yet still other embodiments, the amount of hydrogen sulfate salt of Compound A that can be present in Form A can be in the range of about 95% to about 98%. In some embodiments, the amount of hydrogen sulfate salt of Compound A that can be present in Form A can be ≥90%. In other embodiments, the amount of hydrogen sulfate salt of Compound A that can be present in Form A can be ≥95%. In still other embodiments, the amount of hydrogen sulfate salt of Compound A that can be present in Form A can be ≥98%. In some embodiments, the amount of hydrogen sulfate salt of Compound A that can be present in Form C can be in the range of about 90% to about 100%. In other embodiments, the amount of hydrogen sulfate salt of Compound A that can be present in Form C can be in the range of about 95% to about 100%. In still other embodiments, the amount of hydrogen sulfate salt of Compound A that can be present in Form C can be in the range of about 98% to about 100%. In yet still other embodiments, the amount of hydrogen sulfate salt of Compound A that can be present in Form C can be in the range of about 95% to about 98%. In some embodiments, the amount of hydrogen sulfate salt of Compound A that can be present in Form C can be ≥90%. In other embodiments, the amount of hydrogen sulfate salt of Compound A that can be present in Form C can be ≥95%. In still other embodiments, the amount of hydrogen sulfate salt of Compound A that can be present in Form C can be ≥98%.

The ratio of Compound A to hydrosulfate can vary. Also, the ratio of Compound A to sulfate can vary. In some embodiments, the ratio of Compound A to hydrosulfate (Compound A:hydrosulfate) can be about 1.3:about 1. In some embodiments, the ratio of Compound A to hydrosulfate (Compound A:hydrosulfate) can be about 1.2:about 1. In some embodiments, the ratio of Compound A to hydrosulfate (Compound A:hydrosulfate) can be about 1.1:about 1. In some embodiments, the ratio of Compound A to hydrosulfate (Compound A:hydrosulfate) can be about 1:about 1. In other embodiments, the ratio of Compound A to sulfate (Compound A:sulfate) can be about 2:about 1.

As described herein, Compound A can exist as a variety of salt forms, including Form A, Form C, Form D, Form E and amorphous. Some embodiments described herein include a mixture of Form A and Form C. In other embodiments described herein include a mixture of Form A and amorphous. Other embodiments include a mixture of Form A, Form C and amorphous. Some embodiments include a mixture that includes at least Form A and optionally Form C and/or amorphous.

The amount of Form A that can be in a mixture can vary. In some embodiments, the amount of Form A in a mixture can be greater than 95% based on the total amount of Compound A in the mixture. In some embodiments, the amount of Form A in a mixture can be greater than 85% based on the total amount of Compound A in the mixture. In some embodiments, the amount of Form A in a mixture can be in the range of about 99% to about 80% based on the total amount of Compound A in the mixture.

Various methods can be used to characterize the solid forms described herein. For example, X-ray diffraction, DSC, TGA, IR, TGIR, ¹H NMR and ¹³C NMR. In some embodiments, Form A can be characterized by one or more peaks in an X-ray powder diffraction pattern, wherein the one or more peaks can be selected from a peak in the range of from about 9.4 degrees 2θ to about 9.7 degrees 2θ, a peak in the range of from about 10.2 degrees 2θ to about 10.5 degrees 2θ and a peak in the range of from about 10.9 degrees 2θ to about 11.2 degrees 2θ. In some embodiments, Form A can be characterized by one or more peaks in an X-ray powder diffraction pattern, wherein the one or more peaks is selected from a peak in the range of from about 4.7 degrees 2θ to about 5.0 degrees 2θ, a peak in the range of from about 9.4 degrees 2θ to about 9.7 degrees 2θ, a peak in the range of from about 10.2 degrees 2θ to about 10.5 degrees 2θ, a peak in the range of from about 10.9 degrees 2θ to about 11.2 degrees 2θ, a peak in the range of from about 14.7 degrees 2θ to about 15.0 degrees 2θ, a peak in the range of from about 16.9 degrees 2θ to about 17.2 degrees 2θ, a peak in the range of from about 19.6 degrees 2θ to about 19.9 degrees 2θ, and a peak in the range of from about 20.9 degrees 2θ to about 21.1 degrees 2θ.

In some embodiments, Form A can be characterized by one or more peaks in an X-ray powder diffraction pattern, wherein the one or more peaks can be selected from about 9.56 degrees 2θ±0.2 degrees 2θ, about 10.33 degrees 2θ±0.2 degrees 2θ and about 11.00 degrees 2θ±0.2 degrees 2θ. In some embodiments, Form A can be characterized by one or more peaks in an X-ray powder diffraction pattern, wherein the one or more peaks is selected from about 4.83 degrees 2θ±0.2 degrees 2θ, about 9.56 degrees 2θ±0.2 degrees 2θ, about 10.33 degrees 2θ±0.2 degrees 2θ, about 11.00 degrees 2θ±0.2 degrees 2θ, about 14.87 degrees 2θ±0.2 degrees 2θ, about 17.05 degrees 2θ±0.2 degrees 2θ, about 19.78 degrees 2θ±0.2 degrees 2θ and about 21.00 degrees 2θ±0.2 degrees 2θ. In some embodiments, Form A can be characterized by one or more peaks in an X-ray powder diffraction pattern, wherein the one or more peaks is selected from about 4.83 degrees 2θ±0.2 degrees 2θ, about 6.49 degrees 2θ±9 0.2 degrees 2θ, about 7.36 degrees 2θ±0.2 degrees 2θ, about 9.56 degrees 2θ±0.2 degrees 2θ, about 10.33 degrees 2θ±0.2 degrees 2θ, about 11.00 degrees 2θ±0.2 degrees 2θ, about 11.41 degrees 2θ±0.2 degrees 2θ, about 13.06 degrees 2θ±0.2 degrees 2θ, about 13.79 degrees 2θ±0.2 degrees 2θ, about 14.87 degrees 2θ±0.2 degrees 2θ, about 15.51 degrees 2θ±0.2 degrees 2θ, about 15.89 degrees 2θ±0.2 degrees 2θ, about 16.62 degrees 2θ±0.2 degrees 2θ, about 17.05 degrees 2θ±0.2 degrees 2θ, about 17.66 degrees 2θ±0.2 degrees 2θ, about 18.68 degrees 2θ±0.2 degrees 2θ, about 19.78 degrees 2θ±0.2 degrees 2θ, about 20.21 degrees 2θ±0.2 degrees 2θ, about 21.00 degrees 2θ±0.2 degrees 2θ, about 21.91 degrees 2θ±0.2 degrees 2θ, about 22.91 degrees 2θ±0.2 degrees 2θ, about 23.84 degrees 2θ±0.2 degrees 2θ, about 24.85 degrees 2θ±0.2 degrees 2θ, about 27.34 degrees 2θ±0.2 degrees 2θ and about 28.83 degrees 2θ±0.2 degrees 2θ.

In some embodiments, Form A can be characterized by one or more peaks in an X-ray powder diffraction pattern, wherein the one or more peaks can be selected from about 9.6 degrees 2θ±0.2 degrees 2θ, about 10.3 degrees 2θ±0.2 degrees 2θ and about 11.0 degrees 2θ±0.2 degrees 2θ. In some embodiments, Form A can be characterized by one or more peaks in an X-ray powder diffraction pattern, wherein the one or more peaks is selected from about 4.8 degrees 2θ±0.2 degrees 2θ, about 9.6 degrees 2θ±0.2 degrees 2θ, about 10.3 degrees 2θ±0.2 degrees 2θ, about 11.0 degrees 2θ±0.2 degrees 2θ, about 14.9 degrees 2θ±0.2 degrees 2θ, about 17.1 degrees 2θ±0.2 degrees 2θ, about 19.8 degrees 2θ±0.2 degrees 2θ and about 21.0 degrees 2θ±0.2 degrees 2θ. In some embodiments, Form A can be characterized by one or more peaks in an X-ray powder diffraction pattern, wherein the one or more peaks is selected from about 4.8 degrees 2θ±0.2 degrees 2θ, about 6.5 degrees 2θ±0.2 degrees 2θ, about 7.4 degrees 2θ±0.2 degrees 2θ, about 9.6 degrees 2θ±0.2 degrees 2θ, about 10.3 degrees 2θ±0.2 degrees 2θ, about 11.0 degrees 2θ±0.2 degrees 2θ, about 11.4 degrees 2θ±0.2 degrees 2θ, about 13.1 degrees 2θ±0.2 degrees 2θ, about 13.8 degrees 2θ±0.2 degrees 2θ, about 14.9 degrees 2θ±0.2 degrees 2θ, about 15.5 degrees 2θ±0.2 degrees 2θ, about 15.9 degrees 2θ±0.2 degrees 2θ, about 16.6 degrees 2θ±0.2 degrees 2θ, about 17.1 degrees 2θ±0.2 degrees 2θ, about 17.7 degrees 2θ±0.2 degrees 2θ, about 18.7 degrees 2θ±0.2 degrees 2θ, about 19.8 degrees 2θ±0.2 degrees 2θ, about 20.2 degrees 2θ±0.2 degrees 2θ, about 21.0 degrees 2θ±0.2 degrees 2θ, about 21.9 degrees 2θ±0.2 degrees 2θ, about 22.9 degrees 2θ±0.2 degrees 2θ, about 23.8 degrees 2θ±0.2 degrees 2θ, about 24.9 degrees 2θ±0.2 degrees 2θ, about 27.3 degrees 2θ±0.2 degrees 2θ and about 28.8 degrees 2θ±0.2 degrees 2θ.

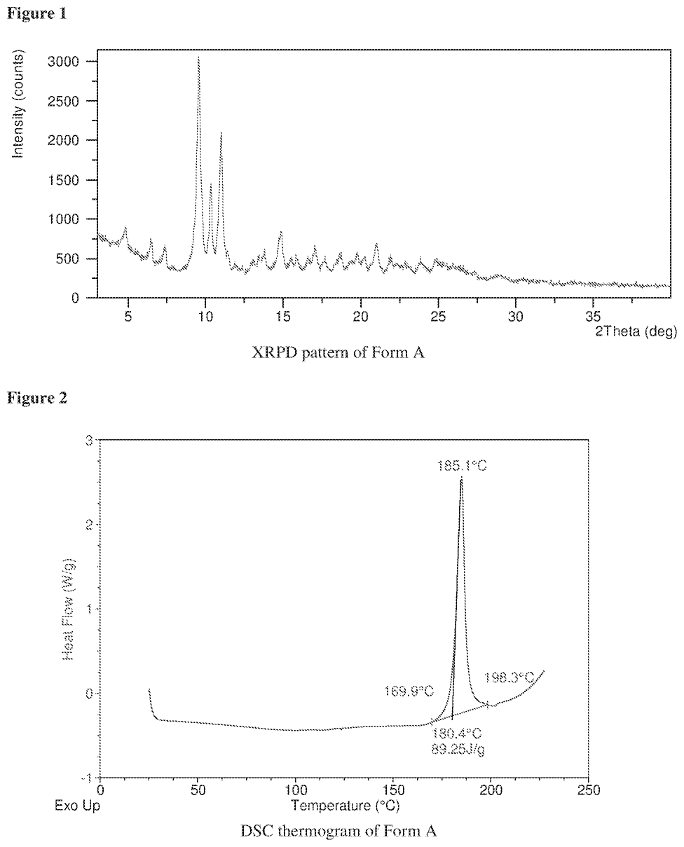

In some embodiments, Form A can exhibit an X-ray powder diffraction pattern as shown in FIG. 1. All XRPD patterns provided herein are measured on a degrees 2-Theta (2θ) scale. It should be understood that the numerical values of the peaks of an X-ray powder diffraction pattern may vary from one machine to another, or from one sample to another, and so the values quoted are not to be construed as absolute, but with an allowable variability, such as ±0.2 degrees two theta (2θ), or more. For example, in some embodiments, the value of an XRPD peak position may vary by up to ±0.2 degrees 2θ while still describing the particular XRPD peak.

In some embodiments, Form A can be characterized by one or more peaks in an X-ray powder diffraction pattern selected from:

| Peak | °2θ | d-spacing [Å] | Relative Intensity [%] |
|---|---|---|---|
| 1 | 4.83 | 18.30 | 10.80 |
| 2 | 6.49 | 13.62 | 9.19 |
| 2 | 7.36 | 12.02 | 8.02 |
| 4 | 9.56 | 9.26 | 100.00 |
| 5 | 10.33 | 8.56 | 39.79 |
| 6 | 11.00 | 8.05 | 65.28 |
| 7 | 11.41 | 7.75 | 9.59 |
| 8 | 13.06 | 6.78 | 4.16 |
| 9 | 13.79 | 6.42 | 9.21 |
| 10 | 14.87 | 5.96 | 19.16 |
| 11 | 15.51 | 5.71 | 6.29 |
| 12 | 15.89 | 5.58 | 7.05 |
| 13 | 16.62 | 5.33 | 6.79 |
| 14 | 17.05 | 5.20 | 12.75 |
| 15 | 17.66 | 5.02 | 6.45 |
| 16 | 18.68 | 4.75 | 8.36 |
| 17 | 19.78 | 4.49 | 10.54 |
| 18 | 20.21 | 4.39 | 7.99 |
| 19 | 21.00 | 4.23 | 13.92 |
| 20 | 21.91 | 4.06 | 8.25 |
| 21 | 22.91 | 3.88 | 5.75 |
| 22 | 23.84 | 3.73 | 7.98 |
| 23 | 24.85 | 3.58 | 7.70 |
| 24 | 27.34 | 3.26 | 2.95 |
| 25 | 28.83 | 3.10 | 1.98 |

Form A can also be characterized by a DSC. In some embodiments, Form A can be characterized by a DSC thermogram of FIG. 2. In some embodiments can be a crystalline hydrosulfate salt of Compound A having a differential scanning calorimetry thermogram corresponding to the representative differential scanning calorimetry thermogram depicted in FIG. 2. In some embodiments, Form A can be characterized by an exotherm at about 185.1° C. In some embodiments, Form A can be characterized by a differential scanning calorimetry thermogram including an exotherm peak at about 185° C. In some embodiments, Form A can be characterized by a differential scanning calorimetry thermogram including an exotherm onset at about 180° C.

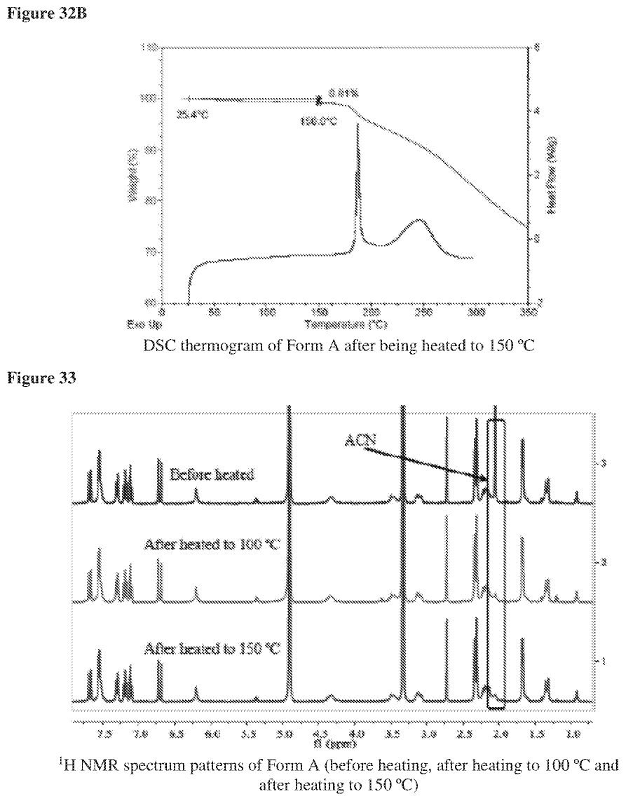

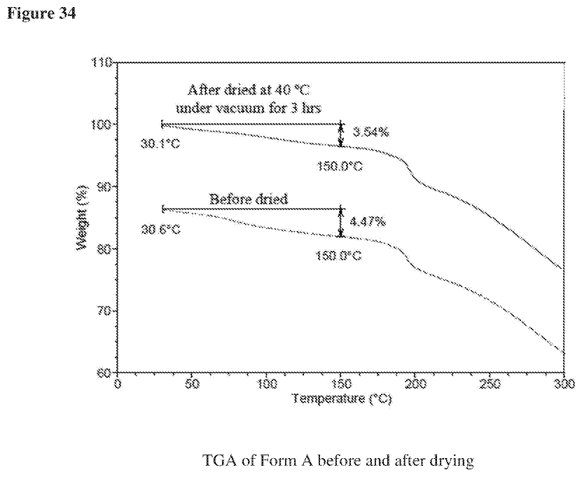

Form A can also be characterized by a thermogravimetric analysis thermogram (TGA). In some embodiments, Form A can be characterized by a TGA thermogram of FIG. 34. In some embodiments, Form A can have a weight loss percent of about 3.54% when heated from about 30° C. to about 150° C. In some embodiments, Form A can have a weight loss percent of about 3.5% when heated from about 30° C. to about 150° C. In some embodiments, Form A can have a weight loss percent in the range of about 2.75% to about 3.75% when heated from about 30° C. to about 150° C. In some embodiments, Form A can have a weight loss percent of about 1.3% when heated from about 25° C. to about 150° C. In some embodiments, Form A can have a weight loss percent of about 0.9% when heated from about 25° C. to about 150° C. In some embodiments, Form A can be characterized by the TGA thermogram depicted in FIG. 32A. In some embodiments, Form A can be characterized by the TGA thermogram depicted in FIG. 32B. In some embodiments, Form A can be characterized by at least one of the TGA thermogram depicted in FIG. 34.

In some embodiments, Form A that has been previously heated to 100° C. has a weight loss of about 1.3% when heated from about 26° C. to about 150° C. In some embodiments, Form A that has been previously heated to 150° C. has a weight loss of about 0.8% when heated from about 25° C. to about 150° C.

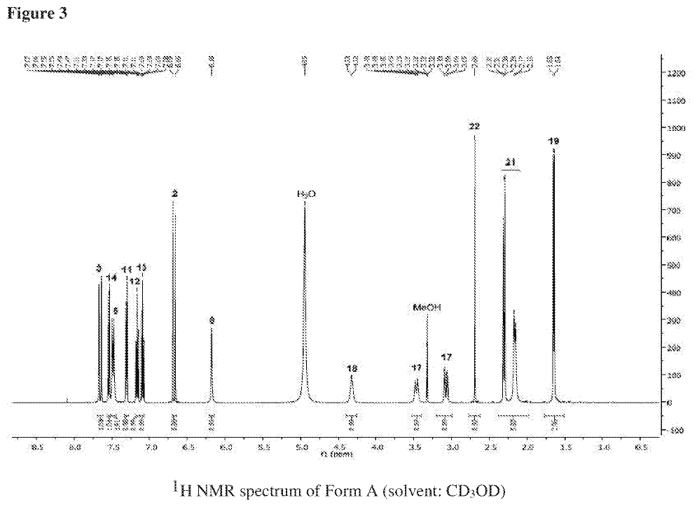

In some embodiments, Form A can be characterized by one or more peaks and/or one or more multiplet of peaks in a ¹H NMR spectrum, wherein the one or more peaks and/or one or more multiplet of peaks can be selected from a peak or a multiplet in the range of 7.69 ppm to 7.61 ppm, a peak or a multiplet in the range of 7.56 ppm to 7.52 ppm, a peak or a multiplet in the range of 7.52 ppm to 7.44 ppm, a peak or a multiplet in the range of 7.33 ppm to 7.28 ppm, a peak or a multiplet in the range of 7.19 ppm to 7.14 ppm, a peak or a multiplet in the range of 7.12 ppm to 7.07 ppm, a peak or a multiplet in the range of 6.70 ppm to 6.65 ppm, a peak or a multiplet in the range of 6.21 ppm to 6.14 ppm, a peak or a multiplet in the range of 4.39 ppm to 4.26 ppm, a peak or a multiplet in the range of 3.53 ppm to 3.40 ppm, a peak or a multiplet in the range of 3.19 ppm to 2.99 ppm, a peak or a multiplet in the range of 2.77 ppm to 2.61 ppm, a peak or a multiplet in the range of 2.38 ppm to 1.97 ppm and a peak or a multiplet in the range of 1.77 ppm to 1.51 ppm. In some embodiments, Form A can be characterized by one or more peaks or one or more multiplet or peaks in a ¹H NMR spectrum, wherein the one or more peaks or one or more multiplets is selected from a peak or a multiplet at about 7.65 ppm, a peak or a multiplet at about 7.54 ppm, a peak or a multiplet at about 7.48 ppm, a peak or a multiplet at about 7.31 ppm, a peak or a multiplet at about 7.17 ppm, a peak or a multiplet at about 7.09 ppm, a peak or a multiplet at about 6.67 ppm, a peak or a multiplet at about 6.18 ppm, a peak or a multiplet at about 4.32 ppm, a peak or a multiplet at about 3.47 ppm, a peak or a multiplet at about 3.07 ppm, a peak or a multiplet at about 2.69 ppm, a peak or a multiplet at about 2.26 ppm and a peak or a multiplet at about 1.64 ppm. In some embodiments, Form A can have the ¹H NMR spectrum of FIG. 3. In some embodiments, including those of this paragraph, the ¹H NMR spectrum can be obtained where the deuterated solvent is CD₃OD. As used herein, “multiplet” refers to a multiplet, a triplet and a doublet as understood by those skilled in the art, unless indicated otherwise.

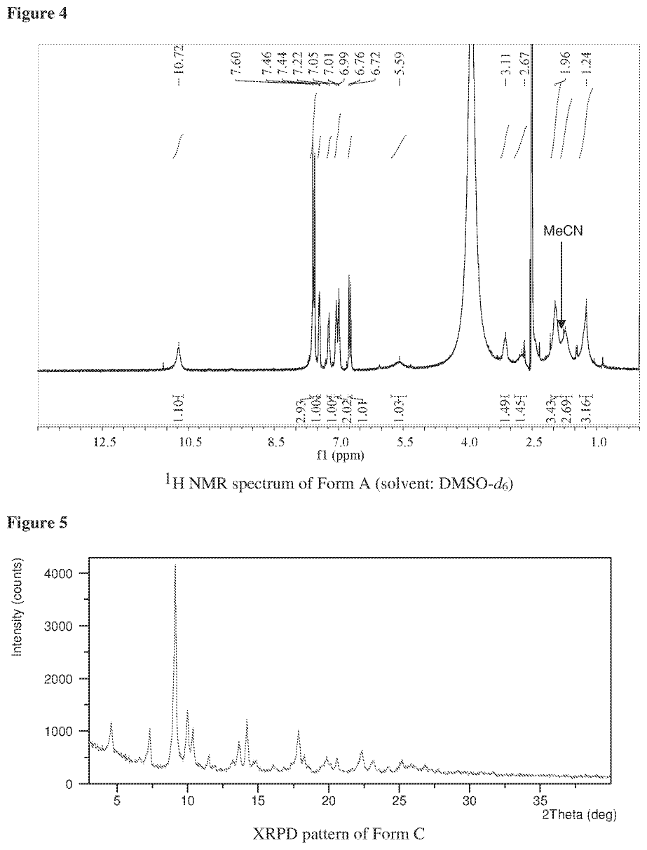

In some embodiments, Form A can be characterized by one or more peaks and/or one or more multiplet of peaks in a ¹H NMR spectrum, wherein the one or more peaks and/or one or more multiplet of peaks can be selected from a peak or a multiplet in the range of 10.76 ppm to 10.68 ppm, a peak or a multiplet in the range of 7.77 ppm to 7.49 ppm, a peak or a multiplet in the range of 7.49 ppm to 7.41 ppm, a peak or a multiplet in the range of 7.14 ppm to 6.92 ppm, a peak or a multiplet in the range of 6.78 ppm to 6.70 ppm, a peak or a multiplet in the range of 5.63 ppm to 5.55 ppm, a peak or a multiplet in the range of 3.15 ppm to 3.07 ppm, a peak or a multiplet in the range of 2.79 ppm to 2.58 ppm, a peak or a multiplet in the range of 2.00 ppm to 1.49 ppm and a peak or a multiplet in the range of 1.28 ppm to 1.20 ppm. In some embodiments, Form A can have the ¹H NMR spectrum of FIG. 4 excluding the peak for acetonitrile (MeCN). In some embodiments, including those of this paragraph, the ¹H NMR spectrum can be obtained where the deuterated solvent is DMSO-d₆.

In some embodiments, Form A can be characterized by one or more peaks and/or one or more multiplets in a ¹H NMR spectrum selected from:

| Chemical Shift | Range | # of H | Type | J, [Hz] |
|---|---|---|---|---|
| 7.65 | 7.69-7.62 | 1 | d | 16.0 |
| 7.54 | 7.56-7.52 | 1 | d | 7.9 |
| 7.48 | 7.52-7.44 | 2 | d | 10.4 |
| 7.31 | 7.33-7.28 | 1 | d | 8.2 |
| 7.17 | 7.19-7.14 | 1 | m | 1.0, 7.1, 8.2 |
| 7.09 | 7.12-7.07 | 1 | m | 1.0, 7.1, 7.9 |
| 6.67 | 6.70-6.65 | 1 | d | 16.0 |
| 6.18 | 6.21-6.14 | 1 | s | — |
| 4.32 | 4.39-4.26 | 1 | m | — |
| 3.47 | 3.53-3.40 | 1 | m | — |
| 3.07 | 3.19-2.99 | 1 | m | — |
| 2.69 | 2.77-2.61 | 1 | br. s | — |
| 2.26 | 2.38-1.97 | 6 | m | — |
| 1.64 | 1.77-1.51 | 3 | d | 6.8 |

wherein the ¹H NMR spectrum of Form A was obtained in CD₃OD.

In other embodiments, Form A can be characterized by one or more peaks and/or one or more multiplets in a ¹H NMR spectrum selected from:

| Chemical Shift | # of H | Type | J, [Hz] |
|---|---|---|---|
| 10.72 | 1 | s | — |
| 7.73-7.53 | 3 | m | — |
| 7.45 | 1 | d | 7.3 |
| 7.22 | 1 | s | — |
| 7.10-6.96 | 2 | m | |
| 6.74 | 1 | d | 16.0 |
| 5.59 | 1 | brs | — |
| 3.11 | 1 | s | — |
| 2.75-2.62 | 1 | m | — |
| 1.96-1.53 | 6 | m | — |
| 1.24 | 2 | s | — |

wherein the ¹H NMR spectrum of Form A was obtained in DMSO-d₆.

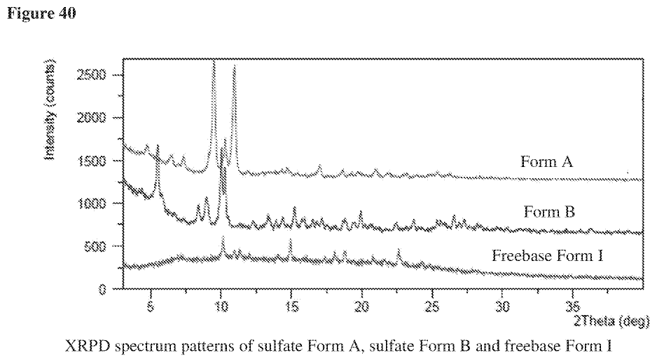

As described herein, hydrosulfate Form B of Compound A can be obtained by slurrying Compound A and H₂SO₄ at about 5° C. for approximately 4 days in acetonitrile:n-heptane (about 1:about 3, v/v). In some embodiment, Form B can have an XRPD peak at about 5.6 degrees 2θ±0.2 degrees 2θ, about 10.0 degrees 2θ±0.2 degrees 2θ and 10.2 degrees 2θ±0.2 degrees 2θ. In some embodiments, Form B can exhibit an X-ray powder diffraction pattern as shown in FIG. 40.

Form C can also be characterized by various methods such as those described herein. In some embodiments, Form C can be characterized by one or more peaks in an X-ray powder diffraction pattern, wherein the one or more peaks can be selected from a peak in the range of from about 9.0 degrees 2θ to about 9.3 degrees 2θ, a peak in the range of from about 9.8 degrees 2θ to about 10.1 degrees 2θ and a peak in the range of from about 14.1 2θ degrees to about 14.4 degrees 2θ. In some embodiments, Form C can be characterized by one or more peaks in an X-ray powder diffraction pattern, wherein the one or more peaks is selected from a peak in the range of from about 4.4 degrees 2θ to about 4.7 degrees 2θ, a peak in the range of from about 7.2 degrees 2θ to about 7.5 degrees 2θ, a peak in the range of from about 9.0 degrees 2θ to about 9.3 degrees 2θ, a peak in the range of from about 9.8 degrees 2θ to about 10.1 degrees 2θ, a peak in the range of from about 10.2 degrees 2θ to about 10.5 degrees 2θ, a peak in the range of from about 11.4 degrees 2θ to about 11.7 degrees 2θ, a peak in the range of from about 13.5 degrees 2θ to about 13.8 degrees 2θ, a peak in the range of from about 14.1 degrees 2θ to about 14.4 degrees 2θ, a peak in the range of from about 17.7 degrees 2θ to about 18.0 degrees 2θ, a peak in the range of from about 18.1 degrees 2θ to about 18.4 degrees 2θ, a peak in the range of from about 19.7 degrees 2θ to about 20.0 degrees 2θ, a peak in the range of from about 20.5 degrees 2θ to about 20.8 degrees 2θ and a peak in the range of from about 22.2 degrees 2θ to about 22.5 degrees 2θ.

In some embodiments, Form C can be characterized by one or more peaks in an X-ray powder diffraction pattern, wherein the one or more peaks can be selected from about 4.6 degrees 2θ±0.2 degrees 2θ, about 7.3 degrees 2θ±0.2 degrees 2θ, about 9.1 degrees 2θ±0.2 degrees 2θ, about 10.0 degrees 2θ±0.2 degrees 2θ, about 10.4 degrees 2θ±0.2 degrees 2θ, about 13.7 degrees 2θ±0.2 degrees 2θ, about 14.2 degrees 2θ±0.2 degrees 2θ, about 17.9 degrees 2θ±0.2 degrees 2θ and about 22.4 degrees 2θ±0.2 degrees 2θ. In some embodiments, Form C can be characterized by one or more peaks in an X-ray powder diffraction pattern, wherein the one or more peaks can be selected from about 9.1 degrees 2θ±0.2 degrees 2θ, about 10.0 degrees 2θ±0.2 degrees 2θ, about 10.4 degrees 2θ±0.2 degrees 2θ, about 14.2 degrees 2θ±0.2 degrees 2θ and about 17.9 degrees 2θ±0.2 degrees 2θ.

In some embodiments, Form C can exhibit an X-ray powder diffraction pattern as shown in FIG. 5. In some embodiments, Form C can be characterized by one or more peaks in an X-ray powder diffraction pattern selected from:

| Peak | °2θ | d-spacing [Å] | Relative Intensity [%] |
|---|---|---|---|
| 1 | 4.58 | 19.3 | 14.40 |
| 2 | 6.59 | 1342 | 2.37 |
| 3 | 7.31 | 12.09 | 17.83 |
| 4 | 9.12 | 9.70 | 100.00 |
| 5 | 9.99 | 8.86 | 27.75 |
| 6 | 10.38 | 8.52 | 20.28 |
| 7 | 11.51 | 7.69 | 7.09 |
| 8 | 11.90 | 7.44 | 1.84 |
| 9 | 13.19 | 6.71 | 4.96 |
| 10 | 13.65 | 6.49 | 14.48 |
| 11 | 14.21 | 6.23 | 24.42 |
| 12 | 14.86 | 5.96 | 5.05 |
| 13 | 16.07 | 5.52 | 3.16 |
| 14 | 17.86 | 4.97 | 20.63 |
| 15 | 18.26 | 4.86 | 7.87 |
| 16 | 18.59 | 4.77 | 2.41 |
| 17 | 19.86 | 4.47 | 6.95 |
| 18 | 20.60 | 4.31 | 6.75 |
| 19 | 22.35 | 3.98 | 9.91 |
| 20 | 23.18 | 3.84 | 6.00 |
| 21 | 24.23 | 3.67 | 2.39 |
| 22 | 25.18 | 3.54 | 5.58 |
| 23 | 25.89 | 3.44 | 3.76 |
| 24 | 26.80 | 3.33 | 3.76 |
| 25 | 27.75 | 3.22 | 1.98 |
| 26 | 31.64 | 2.83 | 1.19 |

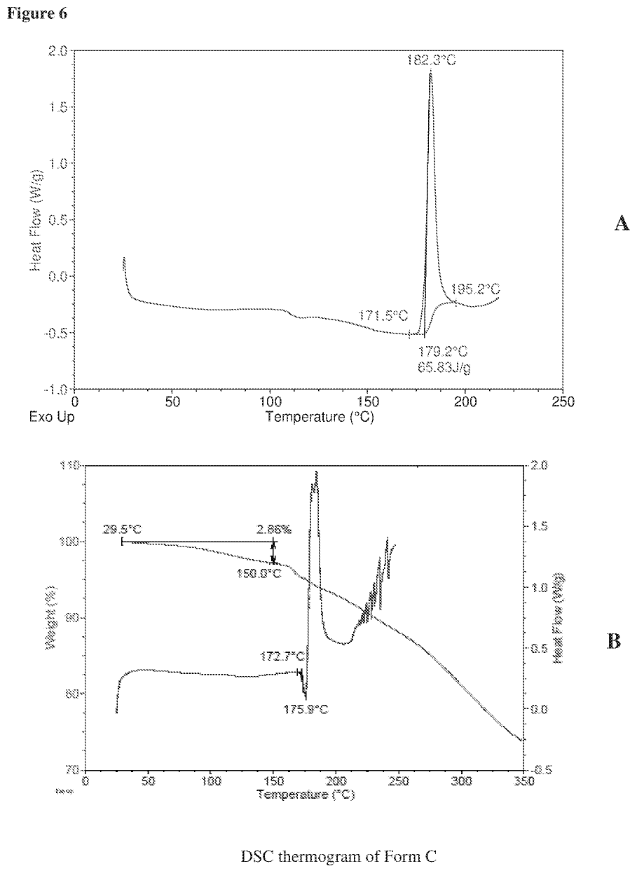

In some embodiments, Form C can be characterized by a differential scanning calorimetry (DSC) thermogram comprising an exotherm at about 182.3° C. In some embodiments, Form C can have a differential scanning calorimetry (DSC) thermogram of FIG. 6A. In some embodiments, provided is a crystalline hydrosulfate salt of Compound A that can have a differential scanning calorimetry thermogram corresponding to the representative differential scanning calorimetry thermogram depicted in FIG. 6A. In other embodiments, Form C, such as a crystalline hydrosulfate salt of Compound A, can have a differential scanning calorimetry (DSC) thermogram of FIG. 6B. In some embodiments, Form C can be characterized by a differential scanning calorimetry thermogram including an exotherm at about 182° C. In some embodiments, Form C can be characterized by a differential scanning calorimetry thermogram including an endotherm at about 176° C. In some embodiments, Form C can have a weight loss percent of about 2.8% when heated from about 30° C. to about 150° C. In some embodiments, Form C can be characterized by the TGA thermogram depicted in FIG. 6B.

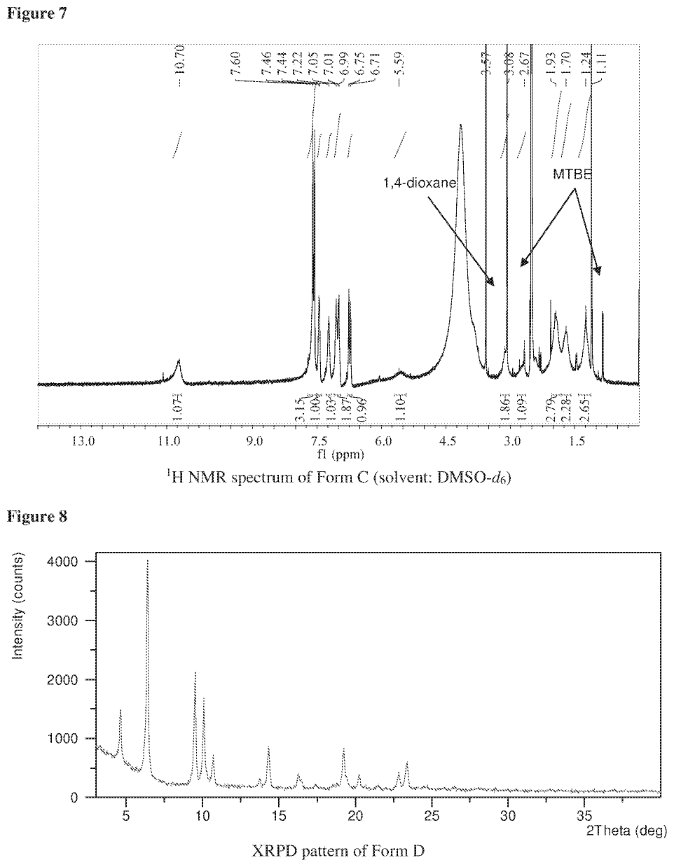

Form C can also be characterized by ¹H NMR. In some embodiments, Form C can be characterized by one or more peaks and/or one or more multiplet of peaks in a ¹H NMR spectrum, wherein the one or more peaks and/or one or more multiplet of peaks can be selected from a peak or a multiplet in the range of 10.74 ppm to 10.66 ppm, a peak or a multiplet in the range of 7.77 ppm to 7.49 ppm, a peak or a multiplet in the range of 7.49 ppm to 7.41 ppm, a peak or a multiplet in the range of 7.26 ppm to 7.18 ppm, a peak or a multiplet in the range of 7.16 ppm to 6.92 ppm, a peak or a multiplet in the range of 6.77 ppm to 6.69 ppm, a peak or a multiplet in the range of 5.63 ppm to 5.55 ppm, a peak or a multiplet in the range of 3.16 ppm to 3.08 ppm, a peak or a multiplet in the range of 2.79 ppm to 2.58 ppm, a peak or a multiplet in the range of 2.00 ppm to 1.49 ppm and a peak or a multiplet in the range of 1.28 ppm to 1.20 ppm. In some embodiments, including those of this paragraph, the ¹H NMR spectrum can be obtained where the deuterated solvent is DMSO-d₆. In some embodiments, Form C can have a ¹H NMR spectrum of FIG. 7 excluding the peaks for 1,4-dioxane and methyl tertiary butyl ether (MTBE). In some embodiments, Form C can be characterized by one or more peaks in a ¹H NMR spectrum selected from:

| Chemical Shift | # of H | Type | J, [Hz] |
|---|---|---|---|
| 10.70 | 1 | s | — |
| 7.73-7.53 | 3 | m | — |
| 7.45 | 1 | d | 6.5 |
| 7.22 | 1 | s | — |
| 7.12-6.96 | 2 | m | — |
| 6.73 | 1 | d | 16.0 |
| 5.59 | 1 | s | — |
| 3.12 | 1 | s | — |
| 2.75-2.62 | 1 | m | — |
| 1.96-1.53 | 6 | m | — |
| 1.24 | 2 | s | — |

wherein the ¹H NMR spectrum of Form C was obtained in DMSO-d₆.

Various other pharmaceutically acceptable salt forms of Compound A can be obtained. Additional pharmaceutically acceptable salt forms of Compound A include, but are not limited to, Form B, Form D and Form E. Various amounts of the hydrosulfate salt of Compound A and the sulfate salt of Compound A can be included in Form B, Form D and Form E. As described herein as an example, the amount of hydrogen sulfate salt of Compound A that can be present in a salt form described herein (such as Form B, Form D and Form E) can be in the range of about 90% to 100%. In some embodiments, a pharmaceutically acceptable salt form of Compound A can be Form D. Form D of Compound A can exhibit an X-ray powder diffraction pattern as shown in FIG. 8.

In some embodiments, Form D can be characterized by one or more peaks in an X-ray powder diffraction pattern, wherein the one or more peaks can be selected from a peak in the range of from about 4.4 degrees 2θ to about 4.8 degrees 2θ, a peak in the range of from about 6.2 degrees 2θ to about 6.6 degrees 2θ, a peak in the range of from about 9.3 degrees 2θ to about 9.7 degrees 2θ, a peak in the range of from about 9.8 degrees 2θ to about 10.2 degrees 2θ, a peak in the range of from about 10.5 degrees 2θ to about 10.9 degrees 2θ, a peak in the range of from about 14.1 degrees 2θ to about 14.5 degrees 2θ, a peak in the range of from about 19.0 degrees 2θ to about 19.4 degrees 2θ and a peak in the range of from about 23.2 degrees 2θ to about 23.6 degrees 2θ. In some embodiments, Form D can be characterized by one or more peaks in an X-ray powder diffraction pattern, wherein the one or more peaks can be selected from a peak in the range of from a peak in the range of from about 6.2 degrees 2θ to about 6.6 degrees 2θ, a peak in the range of from about 9.3 degrees 2θ to about 9.7 degrees 2θ and a peak in the range of from about 9.8 degrees 2θ to about 10.2 degrees 2θ.

In some embodiments, Form D can be characterized by one or more peaks in an X-ray powder diffraction pattern, wherein the one or more peaks can be selected from about 4.6 degrees 2θ±0.2 degrees 2θ, about 6.4 degrees 2θ±0.2 degrees 2θ, about 9.5 degrees 2θ±0.2 degrees 2θ, about 10.0 degrees 2θ±0.2 degrees 2θ, about 10.7 degrees 2θ±0.2 degrees 2θ, about 14.3 degrees 2θ±0.2 degrees 2θ, about 19.2 degrees 2θ±0.2 degrees 2θ and about 23.4 degrees 2θ±0.2 degrees 2θ. In some embodiments, Form D can be characterized by one or more peaks in an X-ray powder diffraction pattern, wherein the one or more peaks can be selected from about 6.4 degrees 2θ±0.2 degrees 2θ, about 9.5 degrees 2θ±0.2 degrees 2θ, and about 10.0 degrees 2θ±0.2 degrees 2θ.

In some embodiments, Form D can be characterized by one or more peaks in an X-ray powder diffraction pattern selected from:

| Peak | °2θ | d-spacing [Å] | Relative Intensity [%] |
|---|---|---|---|
| 1 | 4.62 | 19.12 | 24.34 |
| 2 | 6.39 | 13.84 | 100.00 |
| 3 | 9.51 | 9.30 | 53.33 |
| 4 | 10.08 | 8.77 | 39.75 |
| 5 | 10.69 | 8.28 | 14.64 |
| 6 | 13.74 | 6.44 | 3.58 |
| 7 | 14.32 | 6.18 | 18.79 |
| 8 | 16.27 | 5.45 | 6.78 |
| 9 | 19.22 | 4.62 | 17.75 |
| 10 | 20.25 | 4.39 | 6.47 |
| 11 | 21.46 | 4.14 | 1.54 |
| 12 | 22.84 | 3.89 | 7.52 |
| 13 | 23.38 | 3.81 | 12.10 |

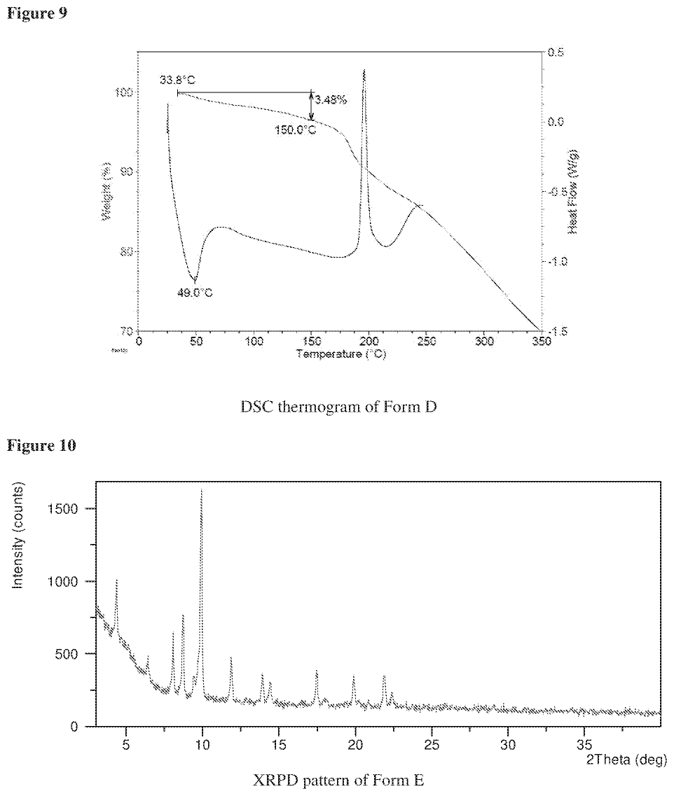

In some embodiments, Form D can have a differential scanning calorimetry (DSC) thermogram of FIG. 9. A differential scanning calorimetry (DSC) thermogram of Form D can be an endotherm at about 49° C. In some embodiments, Form D has an exotherm at about 195° C. In some embodiments, Form D can have a weight loss percent of about 3.5% when heated from about 34° C. to about 150° C. In some embodiments, Form D can be characterized by the TGA thermogram depicted in FIG. 9.

In some embodiments, a pharmaceutically acceptable salt form of Compound A can be Form E. An X-ray powder diffraction pattern of Form E is provided in FIG. 10. In some embodiments, Form E can be characterized by one or more peaks in an X-ray powder diffraction pattern, wherein the one or more peaks can be selected from a peak in the range of from about 4.2 degrees 2θ to about 4.6 degrees 2θ, a peak in the range of from about 7.9 degrees 2θ to about 8.3 degrees 2θ, a peak in the range of from about 8.5 degrees 2θ to about 8.9 degrees 2θ, a peak in the range of from about 9.7 degrees 2θ to about 10.1 degrees 2θ, a peak in the range of from about 11.7 degrees 2θ to about 12.1 degrees 2θ, a peak in the range of from about 13.7 degrees 2θ to about 14.1 degrees 2θ, a peak in the range of from about 17.3 degrees 2θ to about 17.7 degrees 2θ, a peak in the range of from about 19.7 degrees 2θ to about 20.1 degrees 2θ and a peak in the range of from about 21.7 degrees 2θ to about 22.1 degrees 2θ. In some embodiments, Form E can be characterized by one or more peaks in an X-ray powder diffraction pattern, wherein the one or more peaks can be selected from a peak in the range of from about 4.2 degrees 2θ to about 4.6 degrees 2θ, a peak in the range of from about 8.5 degrees 2θ to about 8.9 degrees 2θ, a peak in the range of from about 9.7 degrees 2θ to about 10.1 degrees 2θ and a peak in the range of from about 11.7 degrees 2θ to about 12.1 degrees 2θ.

In some embodiments, Form E can be characterized by one or more peaks in an X-ray powder diffraction pattern, wherein the one or more peaks can be selected from about 4.4 degrees 2θ±0.2 degrees 2θ, about 8.1 degrees 2θ±0.2 degrees 2θ, about 8.7 degrees 2θ±0.2 degrees 2θ, about 9.9 degrees 2θ±0.2 degrees 2θ, about 11.9 degrees 2θ±0.2 degrees 2θ, about 13.9 degrees 2θ±0.2 degrees 2θ, about 17.5 degrees 2θ±0.2 degrees 2θ, about 19.9 degrees 2θ±0.2 degrees 2θ and about 21.9 degrees 2θ±0.2 degrees 2θ. In some embodiments, Form E can be characterized by one or more peaks in an X-ray powder diffraction pattern, wherein the one or more peaks can be selected from about 4.4 degrees 2θ±0.2 degrees 2θ, about 8.7 degrees 2θ±0.2 degrees 2θ, about 9.9 degrees 2θ±0.2 degrees 2θ and about 11.9 degrees 2θ±0.2 degrees 2θ.

In some embodiments, Form E can exhibit an X-ray powder diffraction pattern as shown in FIG. 10. In some embodiments, Form E can be characterized by one or more peaks in an X-ray powder diffraction pattern selected from:

| Peak | °2θ | d-spacing [Å] | Relative Intensity [%] |
|---|---|---|---|
| 1 | 4.36 | 20.25 | 28.61 |
| 2 | 6.40 | 13.81 | 7.52 |
| 3 | 8.06 | 10.97 | 29.88 |
| 4 | 8.71 | 10.16 | 37.08 |
| 5 | 9.91 | 8.92 | 100.00 |
| 6 | 11.87 | 7.45 | 21.58 |
| 7 | 13.92 | 6.36 | 15.17 |
| 8 | 14.41 | 6.15 | 9.39 |
| 9 | 17.47 | 5.08 | 15.71 |
| 10 | 19.89 | 4.46 | 14.22 |
| 11 | 21.89 | 4.06 | 15.77 |
| 12 | 22.39 | 3.97 | 6.56 |

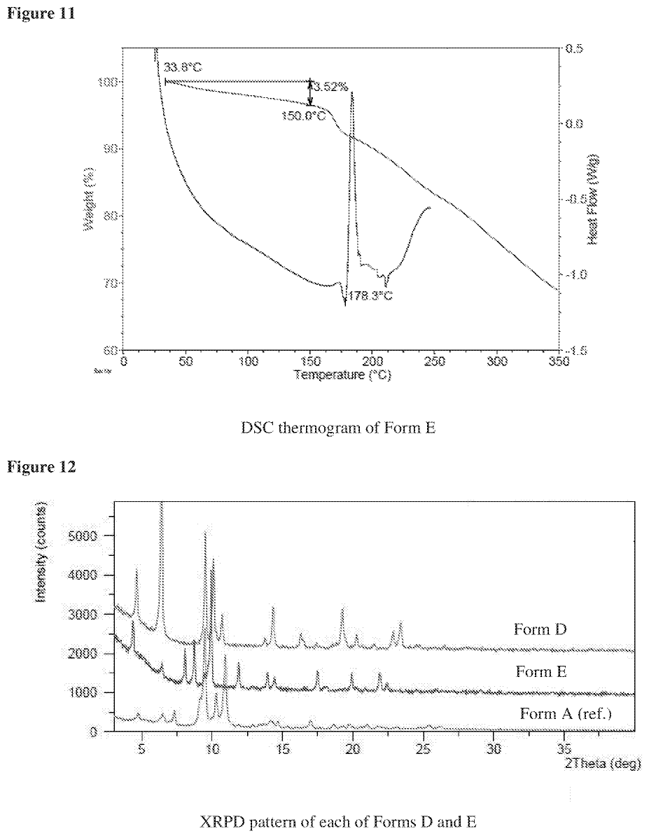

As shown in FIG. 11, Form E can be characterized by a differential scanning calorimetry thermogram that can include an exotherm at about 178° C. In some embodiments, Form E can have a differential scanning calorimetry (DSC) thermogram of FIG. 11. In some embodiments, Form E can have a weight loss percent of about 3.5% when heated from about 34° C. to about 150° C. In some embodiments, Form E can be characterized by the TGA thermogram depicted in FIG. 11.

Various salts of Compound A can be obtained. For example, the following salts can be obtained: HCl, citrate, mesylate, besylate, choline and oxalate.

As described herein, a HCl salt of Compound A can be obtained. In some embodiments, a first HCl salt of Compound A can be characterized by one or more peaks in an X-ray powder diffraction pattern, wherein the one or more peaks can be selected from a peak in the range of from about 5.3 degrees 2θ to about 5.7 degrees 2θ, a peak in the range of from about 8.2 degrees 2θ to about 8.6 degrees 2θ, a peak in the range of from about 8.8 degrees 2θ to about 9.2 degrees 2θ, a peak in the range of from about 9.8 degrees 2θ to about 10.2 degrees 2θ, a peak in the range of from about 10.1 degrees 2θ to about 10.5 degrees 2θ, a peak in the range of from about 13.2 degrees 2θ to about 13.6 degrees 2θ, a peak in the range of from about 14.2 degrees 2θ to about 14.6 degrees 2θ, a peak in the range of from about 15.0 degrees 2θ to about 15.4 degrees 2θ, a peak in the range of from about 17.0 degrees 2θ to about 17.4 degrees 2θ, a peak in the range of from about 18.5 degrees 2θ to about 18.9 degrees 2θ, a peak in the range of from about 19.7 degrees 2θ to about 20.1 degrees 2θ, a peak in the range of from about 23.5 degrees 2θ to about 23.9 degrees 2θ, a peak in the range of from about 26.4 degrees 2θ to about 26.8 degrees 2θ and a peak in the range of from about 27.0 degrees 2θ to about 27.4 degrees 2θ. In some embodiments, a first HCl salt of Compound A can be characterized by one or more peaks in an X-ray powder diffraction pattern, wherein the one or more peaks can be selected from a peak in the range of from about 5.3 degrees 2θ to about 5.7 degrees 2θ, a peak in the range of from about 8.2 degrees 2θ to about 8.6 degrees 2θ, a peak in the range of from about 8.8 degrees 2θ to about 9.2 degrees 2θ, a peak in the range of from about 9.8 degrees 2θ to about 10.2 degrees 2θ, a peak in the range of from about 10.1 degrees 2θ to about 10.5 degrees 2θ and a peak in the range of from about 15.0 degrees 2θ to about 15.4 degrees 2θ. In some embodiments, a second HCl salt of Compound A can be characterized by one or more peaks in an X-ray powder diffraction pattern, wherein the one or more peaks can be selected from a peak in the range of from about 5.3 degrees 2θ to about 5.7 degrees 2θ, a peak in the range of from about 8.8 degrees 2θ to about 9.2 degrees 2θ, a peak in the range of from about 10.8 degrees 2θ to about 11.2 degrees 2θ and a peak in the range of from about 17.0 degrees 2θ to about 17.4 degrees 2θ.

In some embodiments, a first HCl salt of Compound A can be characterized by one or more peaks in an X-ray powder diffraction pattern, wherein the one or more peaks can be selected from about 5.5 degrees 2θ±0.2 degrees 2θ, about 8.4 degrees 2θ±0.2 degrees 2θ, about 9.0 degrees 2θ±0.2 degrees 2θ, about 10.0 degrees 2θ±0.2 degrees 2θ, about 10.3 degrees 2θ±0.2 degrees 2θ, about 13.4 degrees 2θ±0.2 degrees 2θ, about 14.4 degrees 2θ±0.2 degrees 2θ, about 15.2 degrees 2θ±0.2 degrees 2θ, about 17.2 degrees 2θ±0.2 degrees 2θ, about 18.7 degrees 2θ±0.2 degrees 2θ, about 19.9 degrees 2θ±0.2 degrees 2θ, about 23.7 degrees 2θ±0.2 degrees 2θ, about 26.6 degrees 2θ±0.2 degrees 2θ and about 27.2 degrees 2θ±0.2 degrees 2θ. In some embodiments, a first HCl salt of Compound A can be characterized by one or more peaks in an X-ray powder diffraction pattern, wherein the one or more peaks can be selected from about 5.5 degrees 2θ±0.2 degrees 2θ, about 8.4 degrees 2θ±0.2 degrees 2θ, about 9.0 degrees 2θ±0.2 degrees 2θ, about 10.0 degrees 2θ±0.2 degrees 2θ, about 10.3 degrees 2θ±0.2 degrees 2θ and about 15.2 degrees 2θ±0.2 degrees 2θ. In some embodiments, a second HCl salt of Compound A can be characterized by one or more peaks in an X-ray powder diffraction pattern, wherein the one or more peaks can be selected from about 5.5 degrees 2θ±0.2 degrees 2θ, about 9.0 degrees 2θ±0.2 degrees 2θ, about 11.0 degrees 2θ±0.2 degrees 2θ and about 17.2 degrees 2θ±0.2 degrees 2θ.

In some embodiments, a first HCl salt of Compound A can be characterized by one or more peaks in an X-ray powder diffraction pattern selected from:

| Peak | °2θ | d-spacing [Å] | Relative Intensity [%] |
|---|---|---|---|
| 1 | 5.48 | 16.12 | 79.17 |
| 2 | 8.39 | 10.55 | 25.84 |
| 3 | 8.95 | 9.88 | 36.59 |
| 4 | 10.01 | 8.84 | 100.00 |
| 5 | 10.28 | 8.60 | 87.45 |
| 6 | 12.27 | 7.21 | 5.69 |
| 7 | 13.41 | 6.61 | 10.89 |
| 8 | 14.36 | 6.17 | 15.15 |
| 9 | 15.20 | 5.83 | 29.82 |
| 10 | 15.84 | 5.60 | 8.72 |
| 11 | 17.18 | 5.16 | 10.71 |
| 12 | 18.72 | 4.74 | 11.65 |
| 13 | 19.42 | 4.57 | 8.61 |
| 14 | 19.93 | 4.46 | 18.02 |
| 15 | 22.43 | 3.96 | 9.41 |
| 16 | 23.66 | 3.76 | 10.52 |
| 17 | 26.56 | 3.36 | 15.65 |
| 18 | 27.23 | 3.27 | 12.45 |
| 19 | 28.31 | 3.15 | 4.57 |

In other embodiments, a second HCl salt of Compound A can be characterized by one or more peaks in an X-ray powder diffraction pattern selected from:

| Peak | °2θ | d-spacing [Å] | Relative Intensity [%] |
|---|---|---|---|
| 1 | 5.48 | 16.13 | 24.62 |
| 2 | 6.93 | 12.75 | 2.19 |
| 3 | 9.02 | 9.81 | 100.00 |
| 4 | 10.16 | 8.71 | 4.25 |
| 5 | 10.98 | 8.06 | 67.17 |
| 6 | 11.89 | 7.44 | 5.24 |
| 7 | 13.17 | 6.72 | 4.86 |
| 8 | 14.00 | 6.32 | 6.61 |
| 9 | 14.41 | 6.15 | 8.05 |
| 10 | 15.32 | 5.79 | 2.36 |
| 11 | 17.23 | 5.15 | 24.27 |
| 12 | 18.65 | 4.76 | 2.30 |
| 13 | 21.16 | 4.20 | 1.82 |

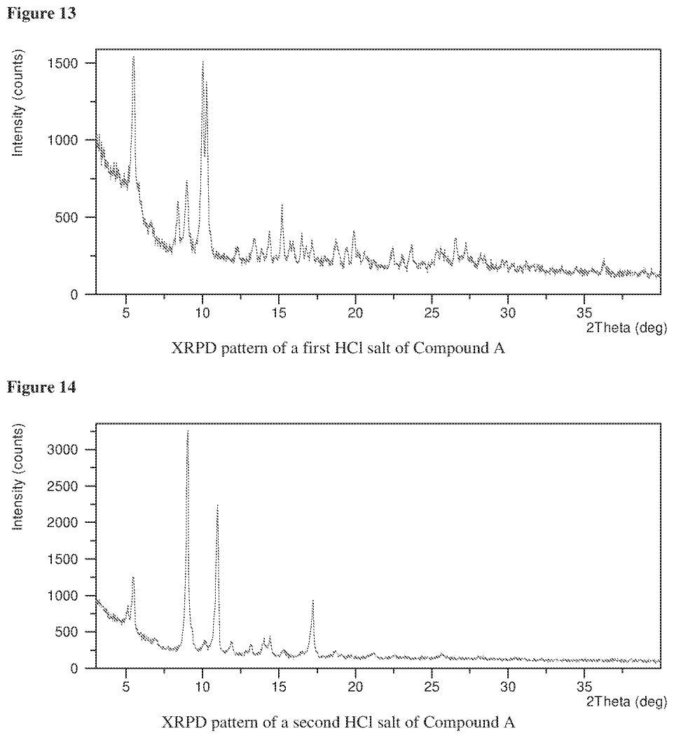

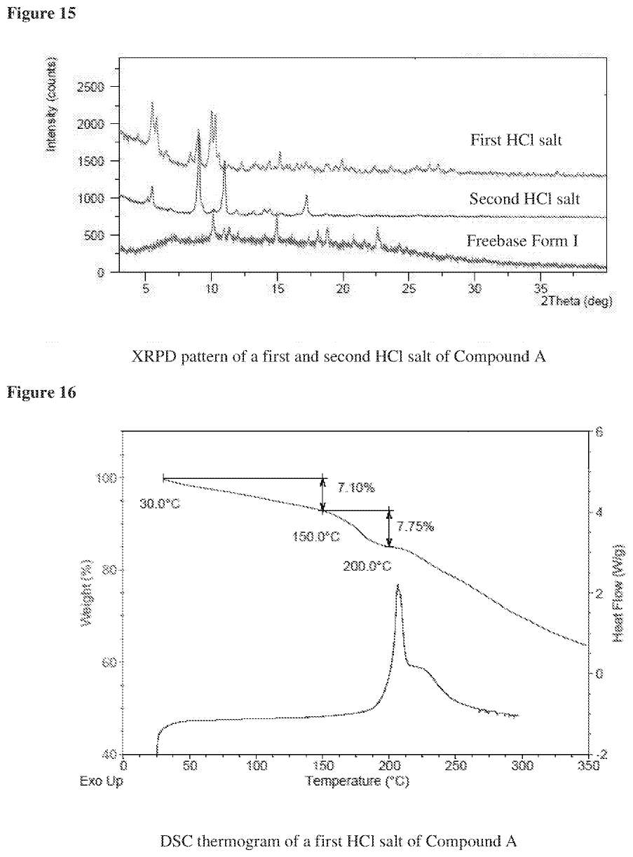

In some embodiments, a first HCl salt of Compound A can exhibit an X-ray powder diffraction pattern as shown in FIG. 13. In some embodiments, a second HCl salt of Compound A can exhibit an X-ray powder diffraction pattern as shown in FIG. 14. In some embodiments, a first HCl salt of Compound A can have a differential scanning calorimetry (DSC) thermogram of FIG. 16. In some embodiments, a first HCl salt of Compound A can have a weight loss percent of about 7.1% when heated from about 30° C. to about 150° C. In some embodiments, a first HCl salt of Compound A can have a weight loss percent of about 7.8% when heated from about 150° C. to about 200° C. In some embodiments, a first HCl salt of Compound A can be characterized by the TGA thermogram depicted in FIG. 16.

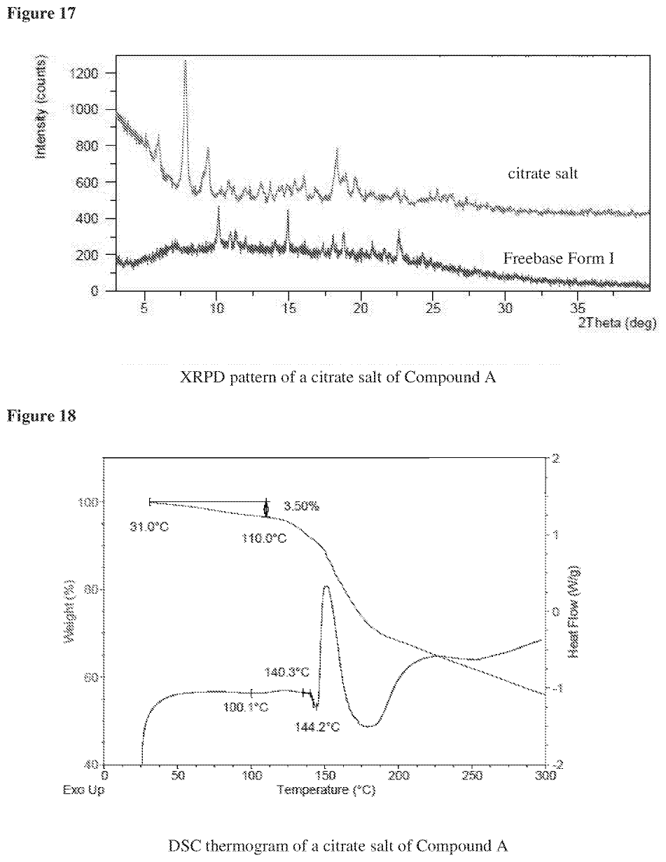

Another salt of Compound A that can be obtained is a citrate salt. In some embodiments, a citrate salt of Compound A can exhibit an X-ray powder diffraction pattern as shown in FIG. 17. In some embodiments, a citrate salt of Compound A can have a differential scanning calorimetry (DSC) thermogram of FIG. 18. In some embodiments, a citrate salt of Compound A can have a weight loss percent of about 3.5% when heated from about 31° C. to about 150° C. In some embodiments, a citrate salt of Compound A can be characterized by the TGA thermogram depicted in FIG. 18.

As described herein, a mesylate salt of Compound A can be obtained. In some embodiments, a first mesylate salt of Compound A can be characterized by one or more peaks in an X-ray powder diffraction pattern, wherein the one or more peaks can be selected from a peak in the range of from about 5.0 degrees 2θ to about 5.4 degrees 2θ, a peak in the range of from about 8.4 degrees 2θ to about 8.8 degrees 2θ, a peak in the range of from about 9.4 degrees 2θ to about 9.8 degrees 2θ, a peak in the range of from about 10.3 degrees 2θ to about 10.7 degrees 2θ and a peak in the range of from about 12.9 degrees 2θ to about 13.3 degrees 2θ. In some embodiments, a first mesylate salt of Compound A can be characterized by one or more peaks in an X-ray powder diffraction pattern, wherein the one or more peaks can be selected from a peak in the range of from about 5.0 degrees 2θ to about 5.4 degrees 2θ, a peak in the range of from about 9.4 degrees 2θ to about 9.8 degrees 2θ and a peak in the range of from about 10.3 degrees 2θ to about 10.7 degrees 2θ. In some embodiments, a second mesylate salt of Compound A can be characterized by one or more peaks in an X-ray powder diffraction pattern, wherein the one or more peaks can be selected from a peak in the range of from about 8.5 degrees 2θ to about 8.9 degrees 2θ, a peak in the range of from about 12.7 degrees 2θ to about 13.1 degrees 2θ and a peak in the range of from about 18.8 degrees 2θ to about 19.2 degrees 2θ.

In some embodiments, a first mesylate salt of Compound A can be characterized by one or more peaks in an X-ray powder diffraction pattern, wherein the one or more peaks can be selected from about 5.2 degrees 2θ±0.2 degrees 2θ, about 8.6 degrees 2θ±0.2 degrees 2θ, about 9.6 degrees 2θ±0.2 degrees 2θ, about 10.5 degrees 2θ±0.2 degrees 2θ and about 13.1 degrees 2θ±0.2 degrees 2θ. In some embodiments, a first mesylate salt of Compound A can be characterized by one or more peaks in an X-ray powder diffraction pattern, wherein the one or more peaks can be selected from about 5.2 degrees 2θ±0.2 degrees 2θ, about 9.6 degrees 2θ±0.2 degrees 2θ and about 10.5 degrees 2θ±0.2 degrees 2θ. In some embodiments, a second mesylate salt of Compound A can be characterized by one or more peaks in an X-ray powder diffraction pattern, wherein the one or more peaks can be selected from about 8.7 degrees 2θ±0.2 degrees 2θ, about 12.9 degrees 2θ±0.2 degrees 2θ and about 19.0 degrees 2θ±0.2 degrees 2θ.

In some embodiments, a first mesylate salt of Compound A can be characterized by one or more peaks in an X-ray powder diffraction pattern selected from:

| Peak | °2θ | d-spacing [Å] | Relative Intensity [%] |
|---|---|---|---|
| 1 | 5.23 | 16.90 | 40.71 |
| 2 | 8.59 | 10.29 | 10.32 |
| 3 | 9.64 | 9.17 | 33.95 |
| 4 | 10.50 | 8.43 | 100.00 |
| 5 | 13.05 | 6.78 | 17.55 |
| 6 | 17.13 | 5.18 | 4.85 |
| 7 | 18.73 | 4.74 | 7.20 |
| 8 | 20.02 | 4.44 | 4.72 |
| 9 | 24.94 | 3.57 | 2.66 |
| 10 | 26.24 | 3.40 | 4.45 |

In other embodiments, a second mesylate salt of Compound A can be characterized by one or more peaks in an X-ray powder diffraction pattern selected from:

| | | | Relative |
|---|---|---|---|
| | | d-spacing | Intensity |
| Peak | °2θ | [Å] | [%] |
| 1 | 4.33 | 20.42 | 9.63 |
| 2 | 8.73 | 10.13 | 100.00 |
| 3 | 11.22 | 7.89 | 6.30 |
| 4 | 12.90 | 6.86 | 14.27 |
| 5 | 14.06 | 6.30 | 3.30 |
| 6 | 15.20 | 5.83 | 6.04 |
| 7 | 18.11 | 4.90 | 5.63 |
| 8 | 18.98 | 4.68 | 20.41 |
| 9 | 20.20 | 4.40 | 9.19 |
| 10 | 22.35 | 3.98 | 5.44 |
| 11 | 22.93 | 3.88 | 5.99 |
| 12 | 23.96 | 3.71 | 3.26 |
| 13 | 25.39 | 3.51 | 5.33 |
| 14 | 26.65 | 3.34 | 2.32 |
| 15 | 29.03 | 3.08 | 0.75 |
| 16 | 33.46 | 2.68 | 2.20 |
| 17 | 39.13 | 2.30 | 1.19 |

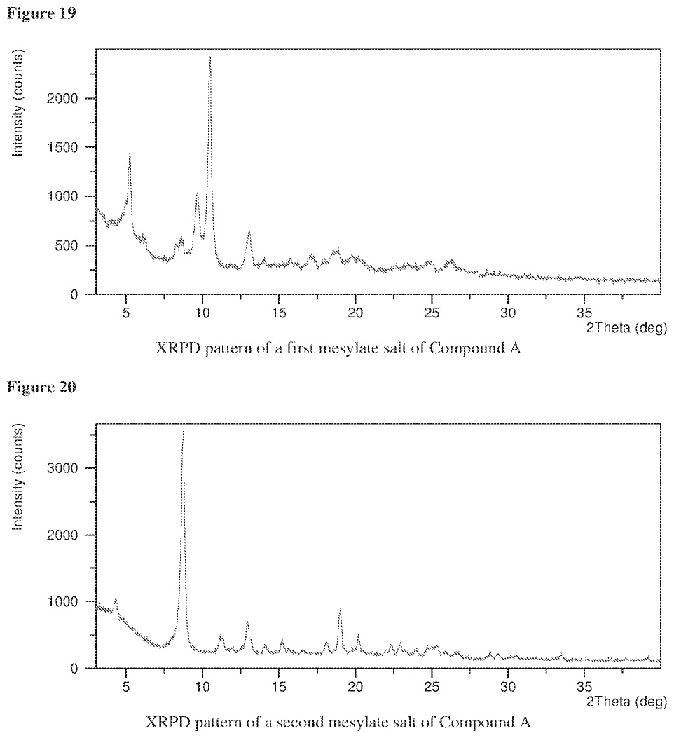

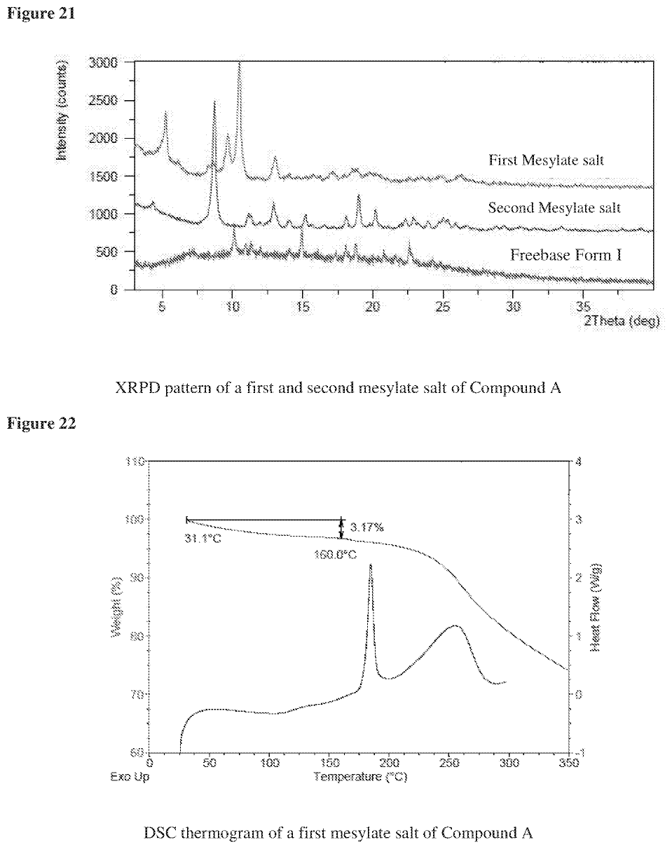

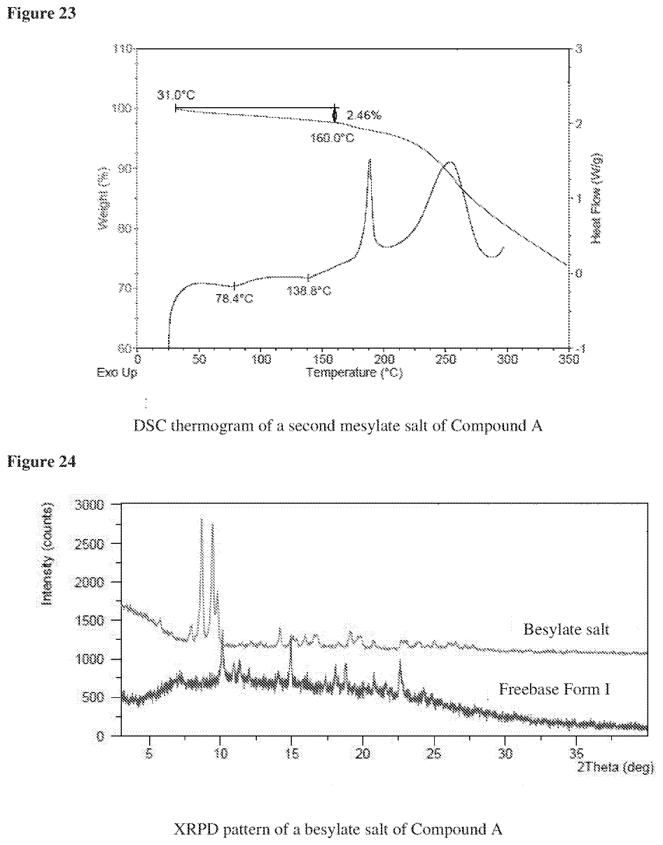

In some embodiments, a first mesylate salt of Compound A can exhibit an X-ray powder diffraction pattern as shown in FIG. 19. In some embodiments, a second mesylate salt of Compound A can exhibit an X-ray powder diffraction pattern as shown in FIG. 20. A mesylate salt of can be characterized by a differential scanning calorimetry thermogram. In some embodiments, a first mesylate salt of Compound A can have a differential scanning calorimetry (DSC) thermogram of FIG. 22. In some embodiments, a second mesylate salt of Compound A can have a differential scanning calorimetry (DSC) thermogram of FIG. 23. In some embodiments, a first mesylate salt of Compound A can have a weight loss percent of about 3.2% when heated from about 31° C. to about 150° C. In some embodiments, a second mesylate salt of Compound A can have a weight loss percent of about 2.5% when heated from about 31° C. to about 150° C. In some embodiments, a first mesylate salt of Compound A can be characterized by a TGA thermogram depicted in FIG. 22. In some embodiments, a second mesylate salt of Compound A can be characterized by a TGA thermogram depicted in FIG. 23.

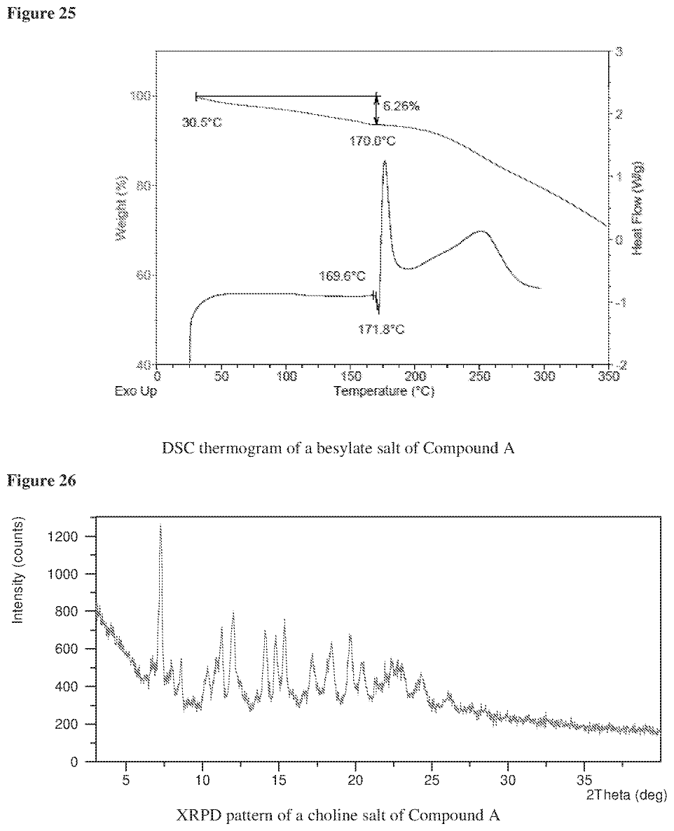

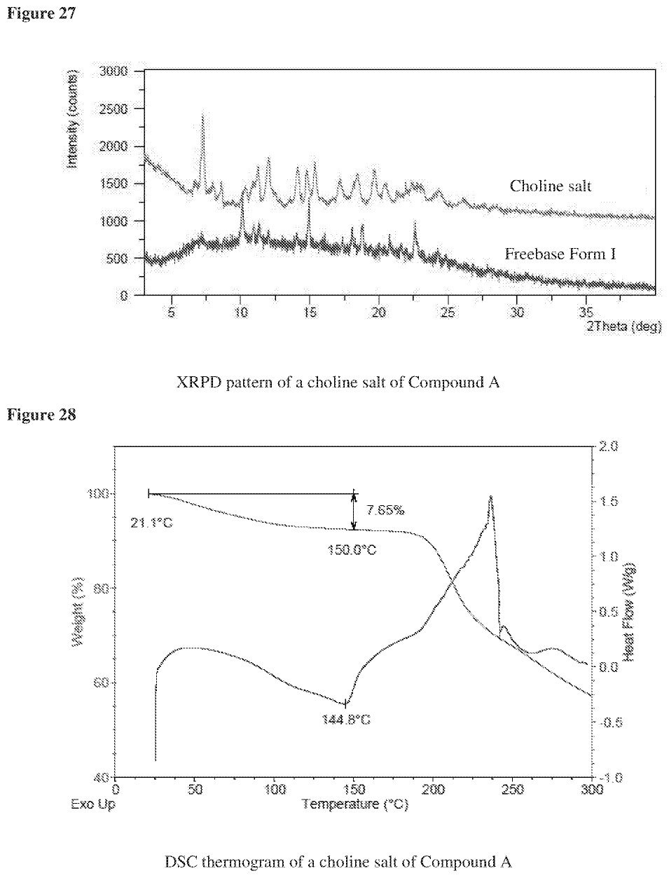

A besylate salt of Compound A can be obtained. In some embodiments, a besylate salt of Compound A can exhibit an X-ray powder diffraction pattern as shown in FIG. 24. In some embodiments, a besylate salt of Compound A can have a differential scanning calorimetry (DSC) thermogram of FIG. 25. In some embodiments, a besylate salt of Compound A can have a weight loss percent of about 6.3% when heated from about 31° C. to about 170° C. In some embodiments, a besylate salt of Compound A can be characterized by the TGA thermogram depicted in FIG. 25.

A choline salt of Compound A can also be obtained. In some embodiments, a choline salt of Compound A can be characterized by one or more peaks in an X-ray powder diffraction pattern, wherein the one or more peaks can be selected from a peak in the range of from about 7.1 degrees 2θ to about 7.5 degrees 2θ, a peak in the range of from about 7.7 degrees 2θ to about 8.1 degrees 2θ, a peak in the range of from about 8.4 degrees 2θ to about 8.8 degrees 2θ, a peak in the range of from about 10.2 degrees 2θ to about 10.6 degrees 2θ, a peak in the range of from about 11.1 degrees 2θ to about 11.5 degrees 2θ, a peak in the range of from about 11.9 degrees 2θ to about 12.3 degrees 2θ, a peak in the range of from about 13.9 degrees 2θ to about 14.3 degrees 2θ, a peak in the range of from about 14.5 degrees 2θ to about 14.9 degrees 2θ, a peak in the range of from about 15.2 degrees 2θ to about 15.6 degrees 2θ, a peak in the range of from about 16.9 degrees 2θ to about 17.3 degrees 2θ, a peak in the range of from about 18.3 degrees 2θ to about 18.7 degrees 2θ, a peak in the range of from about 19.5 degrees 2θ to about 19.9 degrees 2θ, a peak in the range of from about 20.2 degrees 2θ to about 20.6 degrees 2θ, a peak in the range of from about 22.3 degrees 2θ to about 22.7 degrees 2θ and a peak in the range of from about 24.2 degrees 2θ to about 24.6 degrees 2θ. In some embodiments, a choline salt of Compound A can be characterized by one or more peaks in an X-ray powder diffraction pattern, wherein the one or more peaks can be selected from a peak in the range of from about 7.1 degrees 2θ to about 7.5 degrees 2θ, a peak in the range of from about 11.9 degrees 2θ to about 12.3 degrees 2θ, a peak in the range of from about 15.2 degrees 2θ to about 15.6 degrees 2θ and a peak in the range of from about 19.5 degrees 2θ to about 19.9 degrees 2θ.

In some embodiments, a choline salt of Compound A can be characterized by one or more peaks in an X-ray powder diffraction pattern, wherein the one or more peaks can be selected from about 7.3 degrees 2θ±0.2 degrees 2θ, about 7.9 degrees 2θ±0.2 degrees 2θ, about 8.6 degrees 2θ±0.2 degrees 2θ, about 10.4 degrees 2θ±0.2 degrees 2θ, about 11.3 degrees 2θ±0.2 degrees 2θ, about 12.1 degrees 2θ±0.2 degrees 2θ, about 14.1 degrees 2θ±0.2 degrees 2θ, about 14.7 degrees 2θ±0.2 degrees 2θ, about 15.4 degrees 2θ±0.2 degrees 2θ, about 17.1 degrees 2θ±0.2 degrees 2θ, about 18.5 degrees 2θ±0.2 degrees 2θ, about 19.7 degrees 2θ±0.2 degrees 2θ, about 20.4 degrees 2θ±0.2 degrees 2θ, about 22.5 degrees 2θ±0.2 degrees 2θ and about 24.4 degrees 2θ±0.2 degrees 2θ. In some embodiments, a choline salt of Compound A can be characterized by one or more peaks in an X-ray powder diffraction pattern, wherein the one or more peaks can be selected from about 7.3 degrees 2θ±0.2 degrees 2θ, about 12.1 degrees 2θ±0.2 degrees 2θ, about 15.4 degrees 2θ±0.2 degrees 2θ and about 19.7 degrees 2θ±0.2 degrees 2θ.

In some embodiments, a choline salt of Compound A can be characterized by one or more peaks in an X-ray powder diffraction pattern selected from:

| | | | Relative |
|---|---|---|---|
| | | d-spacing | Intensity |
| Peak | °2θ | [Å] | [%] |
| 1 | 7.25 | 12.20 | 100.00 |
| 2 | 7.93 | 11.14 | 14.90 |
| 3 | 8.60 | 10.29 | 25.99 |
| 4 | 10.36 | 8.54 | 20.50 |
| 5 | 11.26 | 7.86 | 47.31 |
| 6 | 12.05 | 7.35 | 53.09 |
| 7 | 14.13 | 6.27 | 42.54 |
| 8 | 14.74 | 6.01 | 37.41 |
| 9 | 15.38 | 5.76 | 50.76 |
| 10 | 17.14 | 5.17 | 28.42 |
| 11 | 18.45 | 4.81 | 37.89 |
| 12 | 19.67 | 4.51 | 42.40 |
| 13 | 20.39 | 4.36 | 25.36 |
| 14 | 22.49 | 3.95 | 22.99 |
| 15 | 24.39 | 3.65 | 16.93 |

In some embodiments, a choline salt of Compound A can exhibit an X-ray powder diffraction pattern as shown in FIG. 26. In some embodiments, a choline salt of Compound A can have a differential scanning calorimetry (DSC) thermogram of FIG. 28. In some embodiments, a choline salt of Compound A can have a weight loss percent of about 7.7% when heated from about 21° C. to about 150° C. In some embodiments, a choline salt of Compound A can be characterized by the TGA thermogram depicted in FIG. 28.

As described herein, an oxalate salt of Compound A can be obtained. In some embodiments, an oxalate salt of Compound can have an XRPD peak at about 9.9 degrees 2θ±0.2 degrees 2θ. In some embodiments, an oxalate sale of Compound A can exhibit an X-ray powder diffraction pattern as shown in FIG. 39.

Amorphous Compound A can be prepared as described in WO 2017/172957, which is hereby incorporated by reference in its entirety. In some embodiments, a crystalline free base of Compound A (Form I) can be characterized by one or more peaks in an X-ray powder diffraction pattern, wherein the one or more peaks can be selected from a peak in the range of from about 9.9 degrees 2θ to about 10.3 degrees 2θ, a peak in the range of from about 11.1 degrees 2θ to about 11.5 degrees 2θ, a peak in the range of from about 14.7 degrees 2θ to about 15.1 degrees 2θ, a peak in the range of from about 18.6 degrees 2θ to about 19.0 degrees 2θ and a peak in the range of from about 22.4 degrees 2θ to about 22.8 degrees 2θ.

In some embodiments, Form I can be characterized by one or more peaks in an X-ray powder diffraction pattern, wherein the one or more peaks can be selected from about 10.1 degrees 2θ±0.2 degrees 2θ, about 11.3 degrees 2θ±0.2 degrees 2θ, about 14.9 degrees 2θ±0.2 degrees 2θ, about 18.8 degrees 2θ±0.2 degrees 2θ and about 22.6 degrees 2θ±0.2 degrees 2θ.

In some embodiments, Form I can be characterized by one or more peaks in an X-ray powder diffraction pattern selected from:

| | | | Relative |
|---|---|---|---|
| | | d-spacing | Intensity |
| Peak | °2θ | [Å] | [%] |
| 1 | 10.12 | 8.74 | 94.29 |
| 2 | 11.33 | 7.81 | 42.12 |
| 3 | 14.93 | 5.93 | 100.00 |
| 4 | 18.78 | 4.73 | 52.86 |
| 5 | 22.59 | 3.94 | 62.38 |

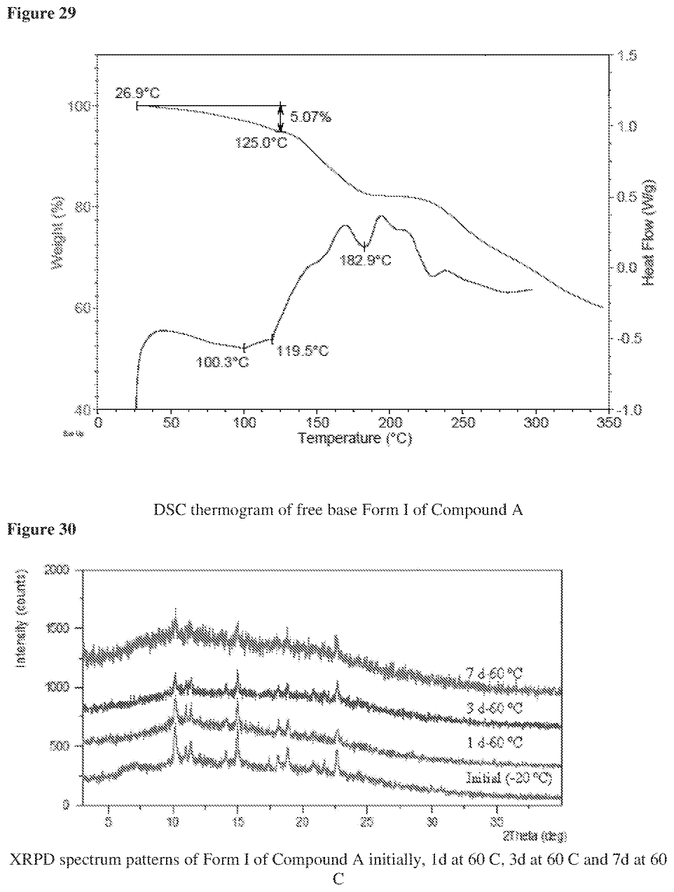

In some embodiments, Form I can have a differential scanning calorimetry (DSC) thermogram of FIG. 29. In some embodiments, Form I can have a weight loss percent of about 5.1% when heated from about 27° C. to about 150° C. In some embodiments, Form I can be characterized by the TGA thermogram depicted in FIG. 29.

Form I was stable in its solid state at 5° C. for 7 days, but degraded upon storage at 40° C. and 60° C. As shown in Table 1, after 7 days at 40° C. and 60° C., Form I degraded approximately 2% and approximately 18%, respectively. Form I also can lose crystallinity upon heating as shown by FIG. 30.

TABLE 1

| | 1 Day | | 3 Days | | 7 Days | |
|---|---|---|---|---|---|---|
| | Area % | Purity vs. | Area % | Purity vs. | Area % | Purity vs. |
| Temp. (° C.) | of A | Initial % | of A | Initial % | of A | Initial % |
| 5 | 98.24 | 99.8 | 98.09 | 99.6 | 98.14 | 99.7 |
| 40 | 97.60 | 99.1 | 96.79 | 98.3 | 96.18 | 97.7 |
| 60 | 93.42 | 94.9 | 88.31 | 89.7 | 80.95 | 82.2 |

In Table 1, “A” represents Compound A, and the data was obtained via HPLC.

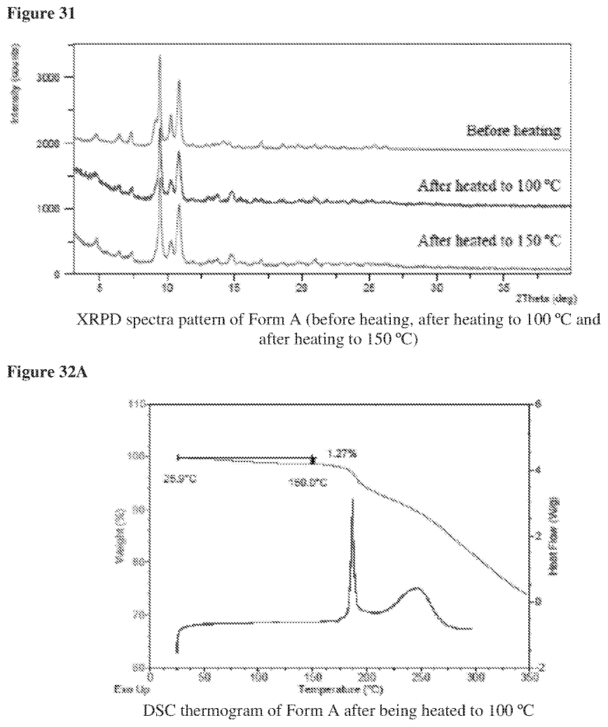

FIG. 31 shows XRPD spectrum of Form A prior to heating, heating to 100° C. and heating to 150° C. Form A retains its crystallinity to a temperature of about 150° C. The stability of Form A is further demonstrated by FIGS. 32A and 32B that shows the DSC spectrum after being heated to 100° C. and 150° C., respectively. FIG. 33 and Table 2 show that Form A can retain a high level of purity at high temperatures. In addition, Form A can be heated to at least to 100° C. that can allow for the removal of any trapped solvent without erosion of purity, which can be beneficial. A comparison of HPLC samples of Form A is shown in Table 2. The purity of initial Form A, Form A after being heated to 100° C., and Form A after being heated to 150° C. are shown. The data suggests that Form A is resistant to heat-promoted degradation. FIG. 34 supports that Form A is resistant to heat-promoted degradation. As shown in FIG. 34, a sample that is heated to a lower temperature (for example, <100° C.) and then to 150° C., and a sample that is heated directly to 150 C shows very little degradation (3.54% and 4.47%, respectively).

TABLE 2

| | | Initial | After | After | |
|---|---|---|---|---|---|
| Peak | | (before | heating | heating | |
| No. | RRT | heating) | to 100° C. | to 150° C. | Identification |
| 2 | 0.55 | 0.67 | 0.67 | 0.76 | degradation |
| 3 | 0.59 | <0.05 | <0.05 | 0.18 | degradation |
| 5 | 0.90 | <0.05 | <0.05 | 0.21 | degradation |
| 6 | 0.94 | <0.05 | <0.05 | 0.16 | degradation |
| 8 | 0.97 | <0.05 | <0.05 | 0.24 | degradation |
| 9 | 1.00 | 99.07 | 99.07 | 97.98 | Compound A |

RRT = relative retention time

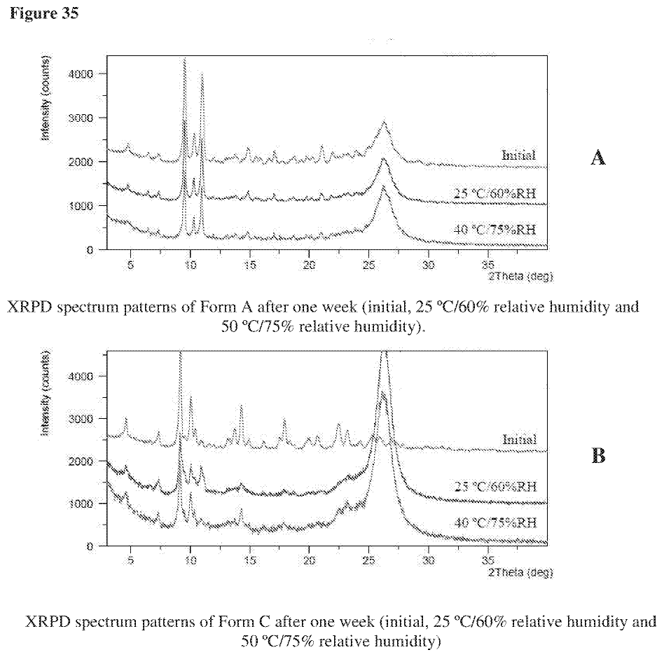

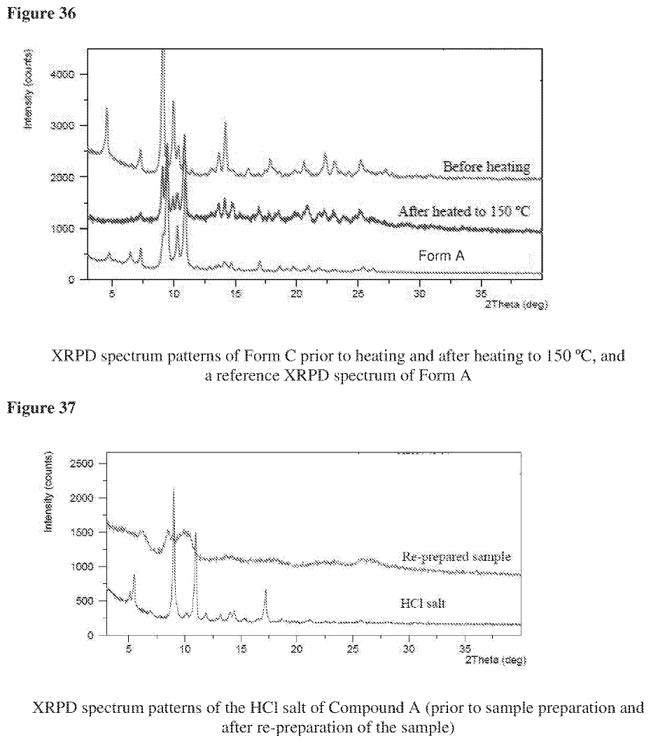

As described herein, Form C is another stable crystalline salt form of Compound A. As shown in FIGS. 35A and 35B, both Form A and Form C are stable after one week at both 25° C. and 60% relative humidity, and 40° C. and 75% relatively humidity. As shown in FIG. 36, Form C partially converts to Form A upon heating to 150° C.

Comparing Form A and Form C that can include the hydrogen sulfate and/or sulfate salt of Compound A to the other salts of Compound A, many of the other salts of Compound A can lose crystallinity, have variable crystallinity and/or have lower purity. As provided in Table 3, the HCl, citrate and besylate salts of Compound A have lower purity compared to Form A.

TABLE 3

| | HPLC Purity (area %) | |
|---|---|---|
| Salt | Before drying | After drying |
| HCl | 98.03 | 98.23 |
| Form A | 98.61 | 98.71 |
| Citrate | 97.64 | 97.69 |
| Mesylate | 98.52 | 98.71 |
| Besylate | 97.90 | 97.75 |

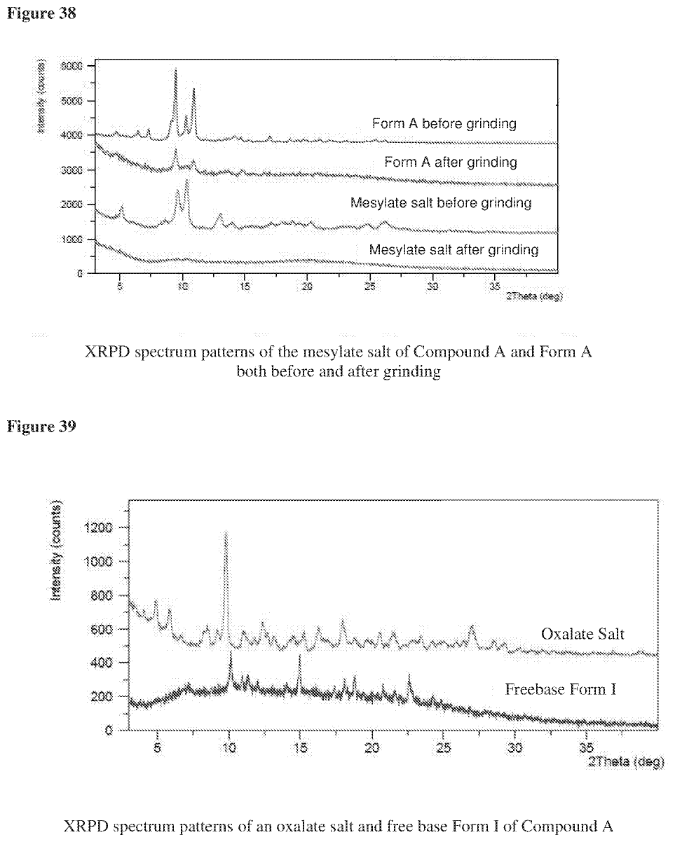

The HCl salt of Compound A demonstrates variable crystallinity during sample preparation as shown by FIG. 37. As shown by the top spectrum, the sharp peaks of the HCl salt of Compound A have flattened out compared to the bottom spectrum. With respect to the mesylate salt of Compound A, the mesylate salt of Compound A shows loss of crystallinity after grinding. As demonstrated by FIG. 38, the peaks from the XRPD spectrum of the mesylate salt of Compound A present before grinding have completely disappeared in the XRPD spectrum after grinding. By comparison, the peaks of Form A (top 2 spectra in FIG. 38) have maintained their sharpness, which indicates that Form A can maintain its crystallinity after grinding (for example, grinding with a mortar and pestle for approximately 5 minutes). The data provide in Table 4 further supports the conclusion that Form A maintains its crystallinity compared to the mesylate salt of Compound A.

TABLE 4

| | Form A | | | Mesylate salt of Compound A | | |
|---|---|---|---|---|---|---|
| | | Heated to | Heated to | | Heated to | Heated to |
| Salt form | Initial | 100° C. | 150° C. | Initial | 100° C. | 150° C. |
| Form change | — | No | No | — | No | No |
| Weight loss | 5.3 | 1.3 | 0.8 | 2.8 | 3.4 | 1.5 |
| (% 150° C.) | | | | | | |
| Endotherm | 135.6, 172.2 | N/A | N/A | N/A | N/A | N/A |
| (° C., peak) | | | | | | |
| Residual | 6.5 | N/A | N/A | | | N/A |
| ACN (wt %) | | | | | | |
| Purity | 99.1 | 99.1 | 98.0 | 98.4 | 98.4 | 97.1 |
| (area %) | | | | | | |

| | | | | |
|---|---|---|---|---|
| | Initial | After grinding for 5 | Initial | After grinding for 5 |
| | | mins. | | mins. |
| Crystallinity | Crystalline | Decreased crystallinity | Crystalline | Amorphous |

Additional salts of Compound A can also be prepared. A choline salt of Compound A was prepared with 97.98% purity. Comparing the purity of Form A to the aforementioned purity of the choline salt of Compound A, Form A has higher purity. The U.S. Food and Drug Administration (FDA) and other agencies stress the importance of impurity control and reproducibility of compounds in drug development. The process is rigorous and helps ensure that an approved drug works correctly and has health benefits that outweigh its known risks. Based on the data provided herein, salts of Compound A, such as the hydrogen sulfate and sulfate salts of Compound A, and salt forms, such as Form A and Form C, described herein are unexpectedly superior for use in a pharmaceutical composition for at least the reasons provided herein.

### Uses and Methods of Treatment

As described herein, a salt of Compound A and/or a salt forms described herein can be used to inhibit the growth of a cell. In some embodiments, the cell is identified as having an estrogen receptor that mediates a growth characteristic of the cell. Growth of a cell can be inhibited by contacting the cell with an effective amount of at least one of the compounds, salts and salt forms described herein, or a pharmaceutical composition as described elsewhere herein. Such contacting of the one or more compounds, salts and salt forms can take place in various ways and locations, including without limitation away from a living subject (e.g., in a laboratory, diagnostic and/or analytical setting) or in proximity to a living subject (e.g., within or on an exterior portion of an animal, e.g., a human). For example, an embodiment provides a method of treating a subject, comprising identifying a subject that is in need of treatment for a disease or condition that is estrogen receptor dependent and/or estrogen receptor mediated and administering to said subject an effective amount of a compound, a salt of Compound A and/or a salt form described elsewhere herein. Another embodiment provides a use of a compound, a salt of Compound A and/or a salt form (as described elsewhere herein), in the manufacture of a medicament for the treatment of a disease or condition that is estrogen receptor alpha dependent and/or estrogen receptor alpha mediated.

Non-limiting examples of diseases or conditions that are estrogen receptor alpha dependent and/or estrogen alpha receptor mediated and thus suitable for treatment using the compounds, salts, salt forms, compositions and methods described herein include breast cancers and gynecological cancers. For example, such diseases or conditions may include one or more of the following: breast cancer, endometrial cancer, ovarian cancer and cervical cancer. An embodiment provides a use of a compound, a salt of Compound A and/or a salt form (as described elsewhere herein), in the manufacture of a medicament for the treatment of breast cancers and gynecological cancers, including for example one or more of the following: breast cancer, endometrial cancer, ovarian cancer and cervical cancer.

Various types of breast cancer are known. In some embodiments, the breast cancer can be ER positive breast cancer. In some embodiments, the breast cancer can be ER positive, HER2-negative breast cancer. In some embodiments, the breast cancer can be local breast cancer (as used herein, “local” breast cancer means the cancer has not spread to other areas of the body). In other embodiments, the breast cancer can be metastatic breast cancer.

At least one point mutation within the Estrogen Receptor 1 (ESR1) that encodes Estrogen receptor alpha (ERα) can exist in breast cancer. The mutation can be in the ligand binding domain (LBD) of ESR1. Examples of mutations can be at an amino acid selected from: A593, S576, G557, R555, L549, A546, E542, L540, D538, Y537, L536, P535, V534, V533, N532, K531, C530, H524, E523, M522, R503, L497, K481, V478, R477, E471, S463, F461, S432, G420, V418, D411, L466, S463, L453, G442, M437, M421, M396, V392, M388, E380, G344, S338, L370, S329, K303, A283, S282, E279, G274, K252, R233, P222, G160, N156, P147, G145, F97, N69, A65, A58 and S47. In some embodiments, one or more mutations can be at an amino acid selected from: D538, Y537, L536, P535, V534, S463, V392 and E380. In some embodiments, one or more mutations can be at an amino acid selected from: D538 and Y537. A non-limiting list of mutations can be selected from: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H. In some embodiments, the mutation can be Y537S. In some embodiments, the mutation can be L536P.

A subject can have a breast cancer that has not been previously treated. In some cases, following breast cancer treatment, a subject can relapse or have reoccurrence of breast cancer. As used herein, the terms “relapse” and “reoccurrence” are used in their normal sense as understood by those skilled in the art. Thus, the breast cancer can be recurrent breast cancer. In some embodiments, the subject has relapsed after a previous treatment for breast cancer. For example, the subject has relapsed after receiving one or more treatments with a SERM, a SERD and/or aromatase inhibitor, such as those described herein.

In some embodiments, the subject had been previously treated with one or more selective ER modulators. For example, subject had been treated previously with one or more selected ER modulators selected from tamoxifen, raloxifene, ospemifene, bazedoxifene, toremifene and lasofoxifene. In some embodiments, the subject had been treated previously with one or more selective ER degraders, such as fulvestrant, elacestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496)), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (Brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid, (E)-3-(4-((2-(4-fluoro-2,6-dimethylbenzoyl)-6-hydroxybenzo[b]thiophen-3-yl)oxy)phenyl)acrylic acid, (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid and/or 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol. In some embodiments, the subject had been treated previously with one or more aromatase inhibitors. The aromatase inhibitors can be a steroidal aromatase inhibitor or a non-steroidal aromatase inhibitor. For example, the one or more aromatase inhibitors can be selected from (exemestane (steroidal aromatase inhibitor), testolactone (steroidal aromatase inhibitor); anastrazole (non-steroidal aromatase inhibitor) and letrazole (non-steroidal aromatase inhibitor).

In some embodiments, the breast cancer can be present in subject, wherein the subject can be a woman. As women approach middle-age, a woman can be in a stage of menopause. In some embodiments, the subject can be a premenopausal woman. In other embodiments, the subject can be a perimenopausal woman. In still other embodiments, the subject can be a menopausal woman. In yet still other embodiments, the subject can be a postmenopausal woman. In other embodiments, the breast cancer can be present in a subject, wherein the subject can be a man. The serum estradiol level of the subject can vary. In some embodiments, the serum estradiol level (E2) of the subject can be in the range of >15 pg/mL to 350 pg/mL. In other embodiments, the serum estradiol level (E2) of the subject can be ≤15 pg/mL. In other embodiments, the serum estradiol level (E2) of the subject can be ≤10 pg/mL.

Compounds, salts of Compound A and/or a salt forms as described elsewhere herein, can be administered to such subjects by a variety of methods. In any of the uses or methods described herein, administration can be by various routes known to those skilled in the art, including without limitation oral, intravenous, intramuscular, topical, subcutaneous, systemic, and/or intraperitoneal administration to a subject in need thereof.

As used herein, the terms “treat,” “treating,” “treatment,” “therapeutic,” and “therapy” do not necessarily mean total cure or abolition of the estrogen receptor dependent and/or estrogen receptor mediated disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of a compound, a salt, a salt form and/or a composition can be the amount needed to prevent, alleviate or ameliorate symptoms of the estrogen receptor dependent and/or estrogen receptor mediated disease or condition, or prolong the survival of the subject being treated. This response may occur in a tissue, system, animal or human and includes alleviation of the signs or symptoms of the estrogen receptor dependent and/or estrogen receptor mediated disease or condition being treated. Determination of an effective amount is well within the capability of those skilled in the art, in view of the disclosure provided herein. The effective amount of the compounds, such as compounds, salts and/or salt forms disclosed herein, required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The amount of a compound, a salt of Compound A and/or a salt form described herein, required for use in treatment will vary not only with the particular compound, salt and/or salt form selected but also with the route of administration, the nature and/or symptoms of the estrogen receptor dependent and/or estrogen receptor mediated disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds, such as compounds, salts and/or salt forms described herein, disclosed herein in amounts that exceed, or even far exceed, the dosage ranges described herein in order to effectively and aggressively treat particularly aggressive estrogen receptor dependent and/or estrogen receptor mediated diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day. The compound, such as a compound, a salt and/or a salt form described herein, may be administered in unit dosage form; for example, containing 1 to 500 mg, 10 to 100 mg or 5 to 50 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a salt of Compound A and/or a salt form described herein, can be determined by comparing their in vitro activity, and in vivo activity in animal models. Such comparison can be done by comparison against an established drug, such as fulvestrant.

Dosage amount and interval may be adjusted individually to provide plasma levels of the active moiety which are sufficient to maintain the modulating effects, or minimal effective concentration (MEC). The MEC will vary for each compound, such as those described herein, but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the estrogen receptor dependent and/or estrogen receptor mediated disease or condition to be treated and to the route of administration. The severity of the estrogen receptor dependent and/or estrogen receptor mediated disease or condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds, such as compounds, salts and/or salt forms described herein, and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds, such as those described herein, in an animal model, such as mice, rats, rabbits, dogs or monkeys, may be determined using known methods. The efficacy of a particular compound, such as those described herein, may be established using several recognized methods, such as in vitro methods, animal models, or human clinical trials. When selecting a model to determine efficacy, the skilled artisan can be guided by the state of the art to choose an appropriate model, dose, route of administration and/or regime.

### Pharmaceutical Compositions

Some embodiments described herein relate to a pharmaceutical composition, that can include an effective amount of a salt of Compound A and/or a salt form described herein (e.g., a hydrogen salt of Compound A, a sulfate salt of Compound A, Form A and/or Form C) and a pharmaceutically acceptable carrier, diluent, excipient or combination thereof.

The term “pharmaceutical composition” refers to a mixture of one or more compounds, such as compounds, salts and/or salt forms described herein, disclosed herein with other chemical components, such as diluents or carriers. The pharmaceutical composition facilitates administration of the compound, such as a compound, a salt and/or a salt form described herein, to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound, such as a compound, a salt and/or a salt form described herein, nor cause appreciable damage or injury to an animal to which delivery of the composition is intended.

As used herein, a “carrier” refers to a compound that facilitates the incorporation of a compound, such as a compound, a salt and/or a salt form described herein, into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds, salts, salt forms and/or compositions described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose.

Multiple techniques of administering a compound, a salt, a salt form and/or a composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections.

One may also administer a compound, a salt, a salt form and/or a composition in a local rather than systemic manner, for example, via injection or implantation of a compound, a salt, a salt form and/or a composition directly into the affected area, often in a depot or sustained release formulation. Furthermore, one may administer a compound, a salt, a salt form and/or a composition in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound, salt and/or salt form described herein formulated in a compatible pharmaceutical carrier may also be prepared, placed in an appropriate container, and labeled for treatment of an indicated condition.

### Examples

Additional embodiments are disclosed in further detail in the following examples, which are not in any way intended to limit the scope of the claims.

As described herein, the amorphous free base of Compound A can be prepared as described in WO 2017/172957. As described in WO 2017/172957, Compound A is an estrogen receptor alpha (ERα) inhibitor.

### Preparation of Form A

### Small-Scale Batch

Into a 20 mL vial was added H₂SO₄ (38.4 μL) followed by the addition of MeCN (8.0 mL). After addition, the mixture was mixed well. The amorphous free base of Compound A (˜200 mg) was added into the vial, and the mixture was slurried at 5° C. at 800 rpm for 1 day. The solid was isolated and dried under vacuum at 50° C. for 30 min. Table 5 provides information regarding the forms of Compound A that were obtained.

TABLE 5

| Peak | | Area (%) | | |
|---|---|---|---|---|
| No. | RRT | Form A | Form B | Identification |
| 1 | 0.51 | 0.83 | 3.60 | impurity |
| 2 | 0.54 | — | 0.23 | impurity |
| 3 | 1.00 | 98.61 | 95.59 | Compound A |
| 4 | 1.13 | 0.14 | 0.08 | impurity |
| 5 | 1.15 | 0.42 | 0.50 | impurity |

Data was obtained via HPLC. HPLC purity of starting Compound A was 98.3% (area)
RRT = relative retention time

### Large-Scale Batch—Procedure 1

Into a 1000-mL reactor was added the amorphous free base of Compound A (˜17.5 g) followed by the addition of MeCN (350.0 mL). The solids were dissolved with stirring at 40° C. at 200 rpm. H₂SO₄ (2.31 mL, 1.05 eq.) was added into MeCN (87.5 mL) to prepare an acid solution. A suspension (43.75 mL) containing Form A seed (˜2.625 g) was added into the amorphous free base of Compound A solution. The acid solution was added into the amorphous free base of Compound A solution over 12 h followed by stirring at 40° C. at 200 rpm for 7 h. The solution was cooled to 20° C. at rate of 0.1° C./min and then stirred at 20° C. at 300 rpm for 4 h. The solution was vacuum filtered, and the cake was washed with MeCN (2×50 mL). The cake was broken into granulates before vacuum drying at 55° C. for 16 h. The granulates were pressed into smaller particulates before vacuum drying at 55° C. for 19 h.

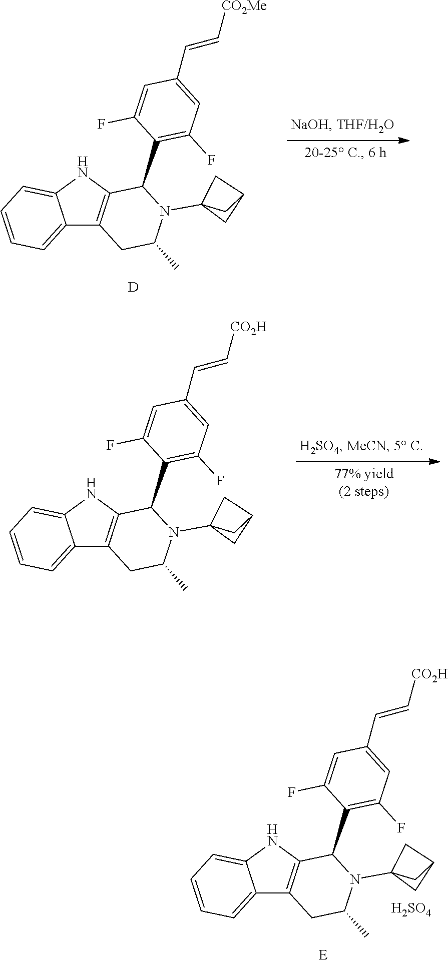

### Large-Scale Batch—Procedure 2

THF (13.3 kg) was added into a 80 L reactor at 15˜25° C. followed by Compound D (7.5 kg) at 15˜25° C. At 15˜25° C., a solution of sodium hydroxide (1.0 kg) in purified water (30.0 kg) was added into the mixture at a rate of 10˜15 kg/h. The mixture was allowed to react at 15˜25° C. After 18˜20 h, the mixture was transferred into a 200 L glass-lined reactor. The mixture was then concentrated at T≤40° C. under reduced pressure until 3.3˜4.0V left. Purified water (7.5 kg) was added into the mixture at T≤40° C. The mixture was concentrated at T≤40° C. under reduced pressure (P≤−0.08 MPa) until 3.3˜4.0V left. The mixture was cooled to 5˜15° C. at a reference rate of 10˜15° C./h. At T≤15° C., the mixture was adjusted pH to 7.5˜8.0 with a solution of sulfuric acid (1.5 kg) in purified water (29.9 kg). Ethyl acetate (23.6 kg) was added into the mixture and stirred for 10˜30 min until the solid dissolved completely by visual check. The mixture was adjusted to 5˜15° C. At T≤15° C., the mixture was adjusted to pH of 6.0˜6.3 with a sulfuric acid solution. At T≤15° C., the mixture was then adjusted to a pH of 5.1˜5.4 with a solution of sulfuric acid (0.4 kg) in purified water (15.0 kg). The mixture was stirred for 15˜30 min at T≤15° C. and then settled for 0.5˜1 h before separation. The aqueous phase was extracted with ethyl acetate (total of ˜50 kg) twice at T≤15° C., and then the mixture was stirred for 15˜30 min and settled for 0.5˜1 h before separation. The mixture in the 80 L glass reactor was concentrated at T≤40° C. under reduced pressure until 14˜16 L left. THF (total of 50 kg) was added into the reactor four times, and the mixture was concentrated at T≤40° C. under reduced pressure until 14˜16 L left. THF (13.4 kg) was added into the mixture, and the mixture was transferred into 200 L Hastelloy reactor. THF (5.7 kg) was added followed by purified water (1.9 kg). The mixture was cooled to 5˜15° C., and a solution of sulfuric acid (1.7 kg) in acetonitrile (28.7 kg) was added into the mixture at a reference rate of 5˜15 kg/h. The mixture was adjusted to 15˜25° C. and maintained for 3˜5 h under stirring. The mixture was filtered with a 220 L Hastelloy agitating filter dryer followed by the additional rinsing with acetonitrile. The solid was dried at T≤40° C. to afford Compound E: (6.9 kg, 76.9% yield) with a purity >99%. ¹H NMR (400 MHz, CD₃OD) δ 7.65 (d, J=16.0 Hz, 1H), 7.54 (d, J=7.9 Hz, 1H), 7.48 (d, J=10.4 Hz, 2H), 7.31 (d, J=8.2 Hz, 1H), 7.19-7.14 (m, 1H), 7.12-7.07 (m, 1H), 6.67 (d, J=16.0 Hz, 1H), 6.18 (s, 1H), 4.39-4.26 (m, 1H), 3.53-3.40 (m, 1H), 3.19-2.99 (m, 1H), 2.69 (br s, 1H), 2.38-1.97 (m, 6H), 1.64 (d, J=6.8 Hz, 3H); MS (ESI) m/z 435.13 [M+H]⁺.

### Preparation of Form C

Into a 100-mL reactor was added the amorphous free base of Compound A (˜1 g) and acetone (10.0 mL). The solution was stirred at room temperature (rt) at 300 rpm. H₂SO₄ (132 μL) as added into acetone (5.0 mL). The H₂SO₄ solution was added into the reactor containing the amorphous free base of Compound A over 20 min, and then stirred at 300 rpm for 17 h at rt. The solution was vacuum filtered, and the cake was washed with acetone (3×10 mL). The cake was vacuum dried at 50° C. for 7 h.

### Preparation of Form D

Form D was prepared in a manner similar as Forms A and C using THF as the solvent.

### Preparation of Form E

Form E was prepared in a manner similar as Forms A and C using a mixture of MeOH/MTBE as the solvent.

### Preparation of Other Salts of Compound A

### HCl Salt

HCl salt of Compound A was obtained via slurrying equimolar amounts of free base of Compound A and HCl at 5° C. for 4 days in acetone:n-heptane (1:3, v/v) and MeCN, respectively. Table 6 provides information regarding the first HCl salt (HCl salt Form A) and the second HCl salt (HCl salt Form B) of Compound A.

TABLE 6

| Peak | | Area (%) | | |
|---|---|---|---|---|
| No. | RRT | Form A | Form B | Identification |
| 1 | 0.51 | 1.27 | 5.79 | impurity |
| 2 | 0.54 | 0.12 | 0.07 | impurity |
| 3 | 0.67 | — | 0.08 | impurity |
| 4 | 0.80 | 0.05 | — | impurity |
| 5 | 1.00 | 98.03 | 93.47 | Compound A |
| 6 | 1.13 | 0.08 | 0.07 | impurity |
| 7 | 1.15 | 0.45 | 0.51 | impurity |

Data was obtained via HPLC. HPLC purity of starting Compound A was 98.3% (area)
RRT = relative retention time

### Citrate Salt

Citrate of Compound A was obtained via slurrying equimolar amount of free base of Compound A and citric acid at 5° C. for 4 days in MeCN. Table 7 provides information regarding the citrate salt of Compound A.

TABLE 7

| Peak | | Area | |
|---|---|---|---|
| No. | RRT | (%) | Identification |
| 1 | 0.51 | 1.55 | impurity |
| 2 | 0.54 | 0.19 | impurity |
| 3 | 0.08 | 0.06 | impurity |
| 4 | 1.00 | 97.64 | Compound A |
| 5 | 1.13 | 0.09 | impurity |
| 6 | 1.15 | 0.47 | impurity |

Data was obtained via HPLC. HPLC purity of starting Compound A was 98.3% (area)
RRT = relative retention time

### Mesylate Salt

Mesylate of Compound A was obtained via slurrying equimolar amount of free base of Compound A and methanesulfonic acid at 5° C. for 4 days in MeCN and acetone:n-heptane (1:3, v/v), respectively. Information regarding the mesylate salts obtained is provided in Table 8.

TABLE 8

| | | Area (%) | | |
|---|---|---|---|---|
| Peak | | Mesylate | Mesylate | |
| No. | RRT | Form A | Form B | Identification |
| 1 | 0.51 | 1.01 | 1.31 | impurity |
| 2 | 0.54 | — | 0.12 | impurity |
| 3 | 0.80 | — | 0.08 | Impurity |
| 4 | 1.00 | 98.52 | 97.97 | Compound A |
| 5 | 1.13 | 0.12 | 0.08 | Impurity |
| 6 | 1.15 | 0.36 | 0.45 | impurity |

Data was obtained via HPLC. HPLC purity of starting Compound A was 98.3% (area)
RRT = relative retention time

### Besylate Salt

Besylate of Compound A was obtained via slurrying equimolar amount of free base of Compound A and benzenesulfonic acid at 5° C. for 4 days in acetone:n-heptane (1:3, v/v). Table 9 provides information regarding the besylate salt obtained.

TABLE 9

| Peak | | Area | |
|---|---|---|---|
| No. | RRT | (%) | Identification |
| 1 | 0.51 | 1.45 | impurity |
| 2 | 0.54 | 0.12 | impurity |
| 3 | 1.00 | 97.90 | Compound A |
| 4 | 1.13 | 0.09 | impurity |
| 5 | 1.15 | 0.44 | impurity |

Data was obtained via HPLC. HPLC purity of starting Compound A was 98.3% (area)
RRT = relative retention time

### Choline Salt

Choline salt of Compound A was obtained via slurrying free base of Compound A and choline at 5° C. for 2 days in MTBE. Information regarding the choline salt obtained is provided in Table 10.

TABLE 10

| Peak | | Area | |
|---|---|---|---|
| No. | RRT | (%) | Identification |
| 1 | 0.27 | 0.12 | impurity |
| 2 | 0.49 | 1.36 | impurity |
| 3 | 0.66 | 0.06 | impurity |
| 4 | 0.80 | 0.11 | impurity |
| 5 | 1.00 | 97.98 | Compound A |
| 6 | 1.15 | 0.37 | impurity |

Data was obtained via HPLC. HPLC purity of starting Compound A was 98.3% (area)
RRT = relative retention time

### Oxalate Salt

Oxalic salt of Compound A was obtained from slurrying free base Compound A and oxalic acid at 5° C. for 2 days in acetone:n-heptane (1:3).

Around 20 mg of free base of Compound A and corresponding salt formers at a 1:1 molar ratio were added in an HPLC glass vial followed by addition of 0.5 mL solvent. After slurry at 1000 rpm at 5° C. for 4 days, the resulting suspension was centrifuged to retrieve solids for further analysis. Additional information regarding the formation of salts of Compound A are provided in Table 11.

TABLE 11

| | Solvent | | |
|---|---|---|---|
| | | | C |
| | | | Acetone: |
| | A | B | n-heptane |
| Co-former | MTBE | ACN | (1:3, v/v) |
| HCl | HCl salt Form A | HCl salt Form B | HCl salt |
| | | | Form A |
| H₂SO₄ | Amorphous | Sulfate Form A | Sulfate |
| | | | Form B |
| H₃PO₄ | Weak crystallinity | Phosphate Form A | Phosphate |
| | | | Form B |
| Citric acid | NCSF | Weak crystalline | Citrate |
| monohydrate | | | Form A |
| L-tartaric acid | NCSF | Weak crystalline | Weak |
| | | | crystalline |
| Acetic acid | NCSF | Clear | Amorphous |
| Succinic acid | NCSF | Succinic acid | NCSF |
| Methanesulfonic | Amorphous | Mesylate Form A | Mesylate |
| acid | | | Form A |
| Benzenesulfonic | Weak crystalline | Besylate Form A | Besylate |
| acid | | | Form A |
| Maleic acid | NCSF | Gel | Gel |

*NCSF indicates no crystalline salt formed

### Characterization Methods

### XRPD

For XRPD analysis, PANalytical X'Pert3 X-ray powder diffractometer was used.

### Parameters for XRPD Test

| Parameters | X′ Pert3 |
|---|---|
| X-Ray wavelength | Cu, Kα; |
| | Kα1 (A): 1.540598 |
| | Kα2 (A): 1.544426 |
| | intensity ratio Kα2/Kα1: 0.50 |
| X-Ray tube setting | 45 kV, 40 mA |
| Divergence slit | 1/8° |
| Scan mode | Continuous |
| Scan range (2θ/°) | 3°~40° |
| Step size (2θ/°) | 0.0263° |
| Scan step time (s) | 46.665 |
| Test time (s) | About 5 mins |

### TGA and DSC

TGA data were collected using a TA Discovery 5500 TGA from TA Instruments. DSC was performed using a TA Discovery 2500 DSC from TA Instruments.

### Parameters for TGA and DSC Test

| Parameters | TGA | DSC |
|---|---|---|
| Method | Ramp | Ramp |
| Sample pan | Aluminum, open | Aluminum, crimped |
| Temperature | RT-Target | 25° C.-Target |
| | temperature | temperature |
| Heating rate | 10° C./min | 10° C./min |
| Purge gas | N₂ | N₂ |

### ¹H NMR

Salt Forms of Compound A were dissolved in DMSO-d₆ (Form A and Form C) or CD₃OD (Form A). The data was acquired using either a 400 MHz Bruker NMR Spectrometer or 500 MHz Inova NMR spectrometer.

### Breast Cancer Cell Proliferation Assay (MCF-7)

MCF7 was expanded and maintained in the medium (Phenol red free DMEM/F12 (Hyclone SH30272.01) NEAA (Gibco 11140-050) Na-pyruvate (Gibco 11360-070) and Re-stripped Charcoal stripped FBS (Gemini 100-119)). The cells were adjusted to a concentration of 3,000 cells per mL in the above media, and the cells were incubated (37° C., 5% CO₂). The following day a 10 point, serial dilution of compounds was added to the cells at a final concentration ranging from 10-0.000005 μM for the test compounds (17β-estradiol was used as a control). Additional cells were plated in 30 wells to serve as the day 1 (pretreatment) comparison. After 5 days of compound exposure, Cell Titer-Glo reagent was added to the cells, and the relative luminescence units (RLUs) of each well was determined. Cell Titer-Glo was also added to 32 μL of medium without cells to obtain a background value. The plates were allowed to incubate at room temperature for 10 minutes to stabilize luminescent signal, and the luminescence signal was recorded with EnSpire. The relative increase in cell number of each sample is determined as follows: (RLU sample-RLU background/RLU estrogen only treated cells-RLU background)×100=% inhibition.

TABLE 12

| | MCF7 IC₅₀ |
|---|---|
| Test Article | (nM) |
| Free base of Compound A | 0.4 |
| Form A | 0.2 |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A pharmaceutically acceptable salt of (E)-3-(4-((1R,3R)-2-(Bicyclo[1.1.1]pentan-1-yl)-3-methyl-2,3,4,9-tetrahydro-1H-pyrido[3,4-b]indol-1-yl)-3,5-difluorophenyl)acrylic acid (Compound A): 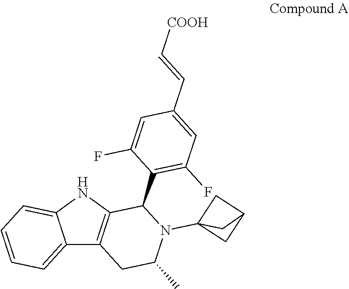 [SMILES: C[C@@H]1Cc2c([nH]c3ccccc23)[C@@H](c2c(F)cc(/C=C/C(=O)O)cc2F)N1C12CC(C1)C2]
wherein the pharmaceutically acceptable salt is a hydrosulfate salt of Compound A.

2. A pharmaceutically acceptable salt of (E)-3-(4-((1R,3R)-2-(Bicyclo[1.1.1]pentan-1-yl)-3-methyl-2,3,4,9-tetrahydro-1H-pyrido[3,4-b]indol-1-yl)-3,5-difluorophenyl)acrylic acid (Compound A): 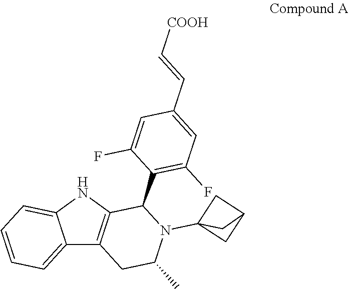 [SMILES: C[C@@H]1Cc2c([nH]c3ccccc23)[C@@H](c2c(F)cc(/C=C/C(=O)O)cc2F)N1C12CC(C1)C2]
wherein the pharmaceutically acceptable salt is a sulfate salt of Compound A.

3. A pharmaceutically acceptable salt form of Compound A comprising the hydrosulfate salt of Compound A; and the sulfate salt of Compound A.

4. The pharmaceutically acceptable salt form of claim 3 consisting essentially of the hydrosulfate salt of Compound A; and the sulfate salt of Compound A.

5. The pharmaceutically acceptable salt form of claim 3, wherein the amount of the hydrosulfate salt of Compound A+the amount of the sulfate salt of Compound A is ≥85% of the pharmaceutically acceptable salt form of Compound A.

6. The pharmaceutically acceptable salt form of claim 3, wherein the amount of the hydrosulfate salt of Compound A+the amount of the sulfate salt of Compound A is ≥98% of the pharmaceutically acceptable salt form of Compound A.

7. The pharmaceutically acceptable salt form of claim 3, wherein the amount of the hydrosulfate salt of Compound A+the amount of the sulfate salt of Compound A is 100% of the pharmaceutically acceptable salt form of Compound A.

8. The pharmaceutically acceptable salt form of any one of claims 3-7, wherein the salt form is Form A.

9. The pharmaceutically acceptable salt form of claim 8, wherein Form A is characterized by one or more peaks in an X-ray powder diffraction pattern, wherein the one or more peaks is selected from a peak in the range of from about 9.4 degrees 2θ to about 9.7 degrees 2θ, a peak in the range of from about 10.2 degrees 2θ to about 10.5 degrees 2θ and a peak in the range of from about 10.9 degrees 2θ to about 11.2 degrees 2θ.

10. The pharmaceutically acceptable salt form of claim 8, wherein Form A is characterized by one or more peaks in an X-ray powder diffraction pattern, wherein the one or more peaks is selected from a peak in the range of from about 4.7 degrees 2θ to about 5.0 degrees 2θ, a peak in the range of from about 9.4 degrees 2θ to about 9.7 degrees 2θ, a peak in the range of from about 10.2 degrees 2θ to about 10.5 degrees 2θ, a peak in the range of from about 10.9 degrees 2θ to about 11.2 degrees 2θ, a peak in the range of from about 14.7 degrees 2θ to about 15.0 degrees 2θ, a peak in the range of from about 16.9 degrees 2θ to about 17.2 degrees 2θ, a peak in the range of from about 19.6 degrees 2θ to about 19.9 degrees 2θ, and a peak in the range of from about 20.9 degrees 2θ to about 21.1 degrees 2θ.

11. The pharmaceutically acceptable salt form of claim 8, wherein Form A is characterized by one or more peaks in an X-ray powder diffraction pattern, wherein the one or more peaks is selected from about 9.6 degrees 2θ±0.2 degrees 2θ, about 10.3 degrees 2θ±0.2 degrees 2θ and about 11.0 degrees 2θ±0.2 degrees 2θ.

12. The pharmaceutically acceptable salt form of claim 8, wherein Form A is characterized by one or more peaks in an X-ray powder diffraction pattern, wherein the one or more peaks is selected from about 4.8 degrees 2θ±0.2 degrees 2θ, about 9.6 degrees 2θ±0.2 degrees 2θ, about 10.3 degrees 2θ±0.2 degrees 2θ, about 11.0 degrees 2θ±0.2 degrees 2θ, about 14.9 degrees 2θ±0.2 degrees 2θ, about 17.1 degrees 2θ±0.2 degrees 2θ, about 19.8 degrees 2θ±0.2 degrees 2θ and about 21.0 degrees 2θ±0.2 degrees 2θ.

13. The pharmaceutically acceptable salt form of claim 8, wherein the Form A is characterized by one or more peaks in an X-ray powder diffraction pattern, wherein the one or more peaks is selected from about 4.8 degrees 2θ±0.2 degrees 2θ, about 6.5 degrees 2θ±0.2 degrees 2θ, about 7.4 degrees 2θ±0.2 degrees 2θ, about 9.6 degrees 2θ±0.2 degrees 2θ, about 10.3 degrees 2θ±0.2 degrees 2θ, about 11.0 degrees 2θ±0.2 degrees 2θ, about 11.4 degrees 2θ±0.2 degrees 2θ, about 13.1 degrees 2θ±0.2 degrees 2θ, about 13.8 degrees 2θ±0.2 degrees 2θ, about 14.9 degrees 2θ±0.2 degrees 2θ, about 15.5 degrees 2θ±0.2 degrees 2θ, about 15.9 degrees 2θ±0.2 degrees 2θ, about 16.6 degrees 2θ±0.2 degrees 2θ, about 17.1 degrees 2θ±0.2 degrees 2θ, about 17.7 degrees 2θ±0.2 degrees 2θ, about 18.7 degrees 2θ±0.2 degrees 2θ, about 19.8 degrees 2θ±0.2 degrees 2θ, about 20.2 degrees 2θ±0.2 degrees 2θ, about 21.0 degrees 2θ±0.2 degrees 2θ, about 21.9 degrees 2θ±0.2 degrees 2θ, about 22.9 degrees 2θ±0.2 degrees 2θ, about 23.8 degrees 2θ±0.2 degrees 2θ, about 24.9 degrees 2θ±0.2 degrees 2θ, about 27.3 degrees 2θ±0.2 degrees 2θ and about 28.8 degrees 2θ±0.2 degrees 2θ.

14. The pharmaceutically acceptable salt form of claim 8, wherein Form A has an X-ray powder diffraction pattern spectrum corresponding to the representative XRPD spectrum depicted in FIG. 1.

15. The pharmaceutically acceptable salt form of claim 8, wherein Form A is characterized by a differential scanning calorimetry (DSC) thermogram comprising an exotherm peak at about 185° C.

16. The pharmaceutically acceptable salt form of claim 8, wherein Form A has a differential scanning calorimetry (DSC) thermogram corresponding to the representative DSC thermogram depicted in FIG. 2.

17. The pharmaceutically acceptable salt form of any one of claims 3-7, wherein the salt form is Form C.

18. The pharmaceutically acceptable salt form of claim 17, wherein Form C is characterized by one or more peaks in an X-ray powder diffraction pattern, wherein the one or more peaks is selected from a peak in the range of from about 9.0 degrees 2θ to about 9.3 degrees 2θ, a peak in the range of from about 9.8 degrees 2θ to about 10.1 degrees 2θ and a peak in the range of from about 14.1 degrees 2θ to about 14.4 degrees 2θ.

19. The pharmaceutically acceptable salt form of claim 17, wherein Form C is characterized by one or more peaks in an X-ray powder diffraction pattern, wherein the one or more peaks is selected from a peak in the range of from about 4.4 degrees 2θ to about 4.7 degrees 2θ, a peak in the range of from about 7.2 degrees 2θ to about 7.5 degrees 2θ, a peak in the range of from about 9.0 degrees 2θ to about 9.3 degrees 2θ, a peak in the range of from about 9.8 degrees 2θ to about 10.1 degrees 2θ, a peak in the range of from about 10.2 degrees 2θ to about 10.5 degrees 2θ, a peak in the range of from about 11.4 degrees 2θ to about 11.7 degrees 2θ, a peak in the range of from about 13.5 degrees 2θ to about 13.8 degrees 2θ, a peak in the range of from about 14.1 degrees 2θ to about 14.4 degrees 2θ, a peak in the range of from about 17.7 degrees 2θ to about 18.0 degrees 2θ, a peak in the range of from about 18.1 degrees 2θ to about 18.4 degrees 2θ, a peak in the range of from about 19.7 degrees 2θ to about 20.0 degrees 2θ, a peak in the range of from about 20.5 degrees 2θ to about 20.8 degrees 2θ and a peak in the range of from about 22.2 degrees 2θ to about 22.5 degrees 2θ.

20. The pharmaceutically acceptable salt form of claim 17, wherein Form C has an X-ray powder diffraction pattern corresponding to the representative XRPD spectrum depicted in FIG. 5.

21. The pharmaceutically acceptable salt form of claim 17, wherein Form C is characterized by a differential scanning calorimetry (DSC) thermogram comprising an exotherm at about 182° C.

22. The pharmaceutically acceptable salt form of claim 17, wherein Form C has a differential scanning calorimetry (DSC) thermogram corresponding to the representative DSC thermogram depicted in FIG. 6.

23. The pharmaceutically acceptable salt form of any one of claims 3-7, wherein the salt form is Form D.

24. The pharmaceutically acceptable salt form of claim 23, wherein Form D is characterized by one or more peaks in an X-ray powder diffraction pattern, wherein the one or more peaks can be selected from a peak in the range of from a peak in the range of from about 6.2 degrees 2θ to about 6.6 degrees 2θ, a peak in the range of from about 9.3 degrees 2θ to about 9.7 degrees 2θ and a peak in the range of from about 9.8 degrees 2θ to about 10.2 degrees 2θ.

25. The pharmaceutically acceptable salt form of claim 23, wherein Form D has an X-ray powder diffraction pattern corresponding to the representative XRPD spectrum depicted in FIG. 8.

26. The pharmaceutically acceptable salt form of claim 23, wherein Form D has a differential scanning calorimetry (DSC) thermogram corresponding to the representative DSC thermogram depicted in FIG. 9.

27. The pharmaceutically acceptable salt form of any one of claims 3-7, wherein the salt form is Form E.

28. The pharmaceutically acceptable salt form of claim 27, wherein Form E is characterized by one or more peaks in an X-ray powder diffraction pattern, wherein the one or more peaks can be selected from a peak in the range of from about 4.2 degrees 2θ to about 4.6 degrees 2θ, a peak in the range of from about 8.5 degrees 2θ to about 8.9 degrees 2θ, a peak in the range of from about 9.7 degrees 2θ to about 10.1 degrees 2θ and a peak in the range of from about 11.7 degrees 2θ to about 12.1 degrees 2θ.

29. The pharmaceutically acceptable salt form of claim 27, wherein Form E has an X-ray powder diffraction pattern corresponding to the representative XRPD spectrum depicted in FIG. 10.

30. The pharmaceutically acceptable salt form of claim 27, wherein Form E has a differential scanning calorimetry (DSC) thermogram corresponding to the representative DSC thermogram depicted in FIG. 11.

31. A pharmaceutically acceptable salt of (E)-3-(4-((1R,3R)-2-(Bicyclo[1.1.1]pentan-1-yl)-3-methyl-2,3,4,9-tetrahydro-1H-pyrido[3,4-b]indol-1-yl)-3,5-difluorophenyl)acrylic acid (Compound A): 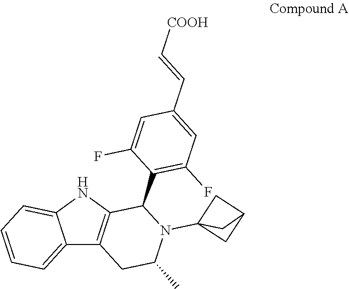 [SMILES: C[C@@H]1Cc2c([nH]c3ccccc23)[C@@H](c2c(F)cc(/C=C/C(=O)O)cc2F)N1C12CC(C1)C2]
wherein the pharmaceutically acceptable salt selected form the group consisting of a HCl salt of Compound A, a citrate salt of Compound A, a mesylate salt of Compound A, a besylate salt of Compound A, a choline salt of Compound A and an oxalate salt of Compound A.

32. The pharmaceutically acceptable salt of claim 31, wherein the pharmaceutically acceptable salt is a HCl salt of Compound A.

33. The pharmaceutically acceptable salt form of claim 32, wherein the HCl salt of Compound A has an X-ray powder diffraction pattern corresponding to the representative XRPD spectrum depicted in FIG. 13.

34. The pharmaceutically acceptable salt form of claim 32, wherein the HCl salt of Compound A has an X-ray powder diffraction pattern corresponding to the representative XRPD spectrum depicted in FIG. 14.

35. The pharmaceutically acceptable salt form of claim 27, wherein the HCl salt of Compound A has a differential scanning calorimetry (DSC) thermogram corresponding to the representative DSC thermogram depicted in FIG. 16.

36. The pharmaceutically acceptable salt of claim 31, wherein the pharmaceutically acceptable salt is a citrate salt of Compound A.

37. The pharmaceutically acceptable salt form of claim 36, wherein the citrate salt of Compound A has an X-ray powder diffraction pattern corresponding to the representative XRPD spectrum depicted in FIG. 17.

38. The pharmaceutically acceptable salt form of claim 36, wherein the citrate salt of Compound A has a differential scanning calorimetry (DSC) thermogram corresponding to the representative DSC thermogram depicted in FIG. 18.

39. The pharmaceutically acceptable salt of claim 31, wherein the pharmaceutically acceptable salt is a mesylate salt of Compound A.

40. The pharmaceutically acceptable salt form of claim 39, wherein the mesylate salt of Compound A has an X-ray powder diffraction pattern corresponding to the representative XRPD spectrum depicted in FIG. 19.

41. The pharmaceutically acceptable salt form of claim 39, wherein the mesylate salt of Compound A is characterized by one or more peaks in an X-ray powder diffraction pattern, wherein the one or more peaks can be selected from a peak in the range of from about 5.0 degrees 2θ to about 5.4 degrees 2θ, a peak in the range of from about 8.4 degrees 2θ to about 8.8 degrees 2θ, a peak in the range of from about 9.4 degrees 2θ to about 9.8 degrees 2θ, a peak in the range of from about 10.3 degrees 2θ to about 10.7 degrees 2θ and a peak in the range of from about 12.9 degrees 2θ to about 13.3 degrees 2θ.

42. The pharmaceutically acceptable salt form of claim 39, wherein the mesylate salt of Compound A is characterized by one or more peaks in an X-ray powder diffraction pattern, wherein the one or more peaks can be selected from a peak in the range of from about 5.0 degrees 2θ to about 5.4 degrees 2θ, a peak in the range of from about 9.4 degrees 2θ to about 9.8 degrees 2θ and a peak in the range of from about 10.3 degrees 2θ to about 10.7 degrees 2θ.

43. The pharmaceutically acceptable salt form of claim 39, wherein the mesylate salt of Compound A is characterized by one or more peaks in an X-ray powder diffraction pattern, wherein the one or more peaks can be selected from a peak in the range of from about 8.5 degrees 2θ to about 8.9 degrees 2θ, a peak in the range of from about 12.7 degrees 2θ to about 13.1 degrees 2θ and a peak in the range of from about 18.8 degrees 2θ to about 19.2 degrees 2θ.

44. The pharmaceutically acceptable salt form of claim 39, wherein the mesylate salt of Compound A is characterized by one or more peaks in an X-ray powder diffraction pattern, wherein the one or more peaks can be selected from about 5.2 degrees 2θ±0.2 degrees 2θ, about 8.6 degrees 2θ±0.2 degrees 2θ, about 9.6 degrees 2θ±0.2 degrees 2θ, about 10.5 degrees 2θ±0.2 degrees 2θ and about 13.1 degrees 2θ±0.2 degrees 2θ.

45. The pharmaceutically acceptable salt form of claim 39, wherein the mesylate salt of Compound A is characterized by one or more peaks in an X-ray powder diffraction pattern, wherein the one or more peaks can be selected from about 8.7 degrees 2θ±0.2 degrees 2θ, about 12.9 degrees 2θ±0.2 degrees 2θ and about 19.0 degrees 2θ±0.2 degrees 2θ.

46. The pharmaceutically acceptable salt form of claim 39, wherein the mesylate salt of Compound A has an X-ray powder diffraction pattern corresponding to the representative XRPD spectrum depicted in FIG. 20.

47. The pharmaceutically acceptable salt form of claim 39, wherein the mesylate salt of Compound A has a differential scanning calorimetry (DSC) thermogram corresponding to the representative DSC thermogram depicted in FIG. 22.

48. The pharmaceutically acceptable salt form of claim 39, wherein the mesylate salt of Compound A has a differential scanning calorimetry (DSC) thermogram corresponding to the representative DSC thermogram depicted in FIG. 23.

49. The pharmaceutically acceptable salt of claim 31, wherein the pharmaceutically acceptable salt is a besylate salt of Compound A.

50. The pharmaceutically acceptable salt form of claim 49, wherein the besylate salt of Compound A has an X-ray powder diffraction pattern corresponding to the representative XRPD spectrum depicted in FIG. 24.

51. The pharmaceutically acceptable salt form of claim 49, wherein the besylate salt of Compound A has a differential scanning calorimetry (DSC) thermogram corresponding to the representative DSC thermogram depicted in FIG. 25.

52. The pharmaceutically acceptable salt of claim 31, wherein the pharmaceutically acceptable salt is a choline salt of Compound A.

53. The pharmaceutically acceptable salt form of claim 52, wherein the choline salt of Compound A has an X-ray powder diffraction pattern corresponding to the representative XRPD spectrum depicted in FIG. 26.

54. The pharmaceutically acceptable salt form of claim 52, wherein the choline salt of Compound A has a differential scanning calorimetry (DSC) thermogram corresponding to the representative DSC thermogram depicted in FIG. 28.

55. The pharmaceutically acceptable salt of claim 31, wherein the pharmaceutically acceptable salt is an oxalate salt of Compound A.

56. The pharmaceutically acceptable salt of any one of claims 3-7 and 31-55 wherein the salt form is substantially chemically pure.

57. The pharmaceutically acceptable salt of any one of claims 3-7 and 31-55 wherein the salt form is substantially physically pure.

58. Crystalline (E)-3-(4-((1R,3R)-2-(Bicyclo[1.1.1]pentan-1-yl)-3-methyl-2,3,4,9-tetrahydro-1H-pyrido[3,4-b]indol-1-yl)-3,5-difluorophenyl)acrylic acid (Compound A):

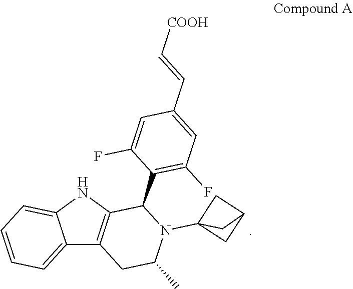 [SMILES: C[C@@H]1Cc2c([nH]c3ccccc23)[C@@H](c2c(F)cc(/C=C/C(=O)O)cc2F)N1C12CC(C1)C2]

59. The crystalline Compound A of claim 58, having a differential scanning calorimetry (DSC) thermogram corresponding to the representative DSC thermogram depicted in FIG. 29.

60. A mixture comprising an amount of pharmaceutically acceptable salt Form A, an amount of pharmaceutically acceptable salt Form C and an amount of amorphous Compound A.

61. A mixture comprising an amount of pharmaceutically acceptable salt Form A and an amount of amorphous Compound A.

62. A mixture comprising an amount of pharmaceutically acceptable salt Form C and an amount of amorphous Compound A.

63. A pharmaceutical composition comprising an effective amount of the pharmaceutically acceptable salt of any one of claims 1-57, and a pharmaceutically acceptable carrier, diluent, excipient or combination thereof.

64. The pharmaceutical composition of claim 63, further comprising an amount of amorphous Compound A.

65. The pharmaceutical composition of claim 63 or 64, further comprising an amount of the free base of Compound A.

66. A method of inhibiting the growth of a cell, comprising
identifying a cell having an estrogen receptor alpha that mediates a growth characteristic of the cell; and
contacting the cell with an effective amount of the pharmaceutically acceptable salt of any one of claims 1-57, a mixture of any one of claims 60-62, or the pharmaceutical composition of any one of claims 63-65.

67. A method of treatment, the method comprising
identifying a subject that is in need of treatment for a disease or condition that is estrogen receptor alpha dependent and/or estrogen receptor alpha mediated; and
administering to said subject an effective amount of the pharmaceutically acceptable salt of any one of claims 1-57, a mixture of any one of claims 60-62, or the pharmaceutical composition of any one of claims 63-65.

68. Use of the pharmaceutically acceptable salt of any one of claims 1-57, a mixture of any one of claims 60-62, or the pharmaceutical composition of any one of claims 63-65, in the manufacture of a medicament for the treatment of a disease or condition that is estrogen receptor alpha dependent and/or estrogen receptor alpha mediated.

69. The method of claim 67 or the use of claim 68, wherein the disease or condition is selected from the group consisting of a breast cancer and a gynecological cancer.

70. The method of claim 67 or the use of claim 68, wherein the disease or condition is selected from the group consisting of breast cancer, endometrial cancer, ovarian cancer, and cervical cancer.

71. The method of claim 67 or the use of claim 68, wherein the disease or condition is breast cancer.

72. The method or use of claim 71, wherein the breast cancer is ER positive breast cancer.

73. The method or use of claim 71, wherein the breast cancer is ER positive/HER2-negative breast cancer.

74. The method or use of claim 71, wherein the breast cancer is local breast cancer.

75. The method or use of claim 71, wherein the breast cancer is metastatic breast cancer.

76. The method or use of claim 71, wherein the breast cancer is recurrent breast cancer.

77. The method or use of claim 71, wherein the breast cancer has been previously treated with an endocrine therapy.
